SEQUENCE LISTING (17 sequences)

SEQ ID NO: 1 - protein, 16 aa, synthetic construct
CIVSFAGGTSYGKLTF

SEQ ID NO: 2 - protein, 15 aa, synthetic construct
CIGNAGGTSYGKLTF

SEQ ID NO: 3 - protein, 16 aa, synthetic construct
CISQKSGGTSYGKLTF

SEQ ID NO: 4 - protein, 16 aa, synthetic construct
CIVSIAGGTSYGKLTF

SEQ ID NO: 5 - protein, 16 aa, synthetic construct
CTLSLAGGTSYGKLTF

SEQ ID NO: 6 - protein, 13 aa, synthetic construct
CASSLTGGYGYTF

SEQ ID NO: 7 - protein, 12 aa, synthetic construct
CASSLSNYGYTF

SEQ ID NO: 8 - protein, 13 aa, synthetic construct
CASSLTGGYEQYF

SEQ ID NO: 9 - protein, 13 aa, synthetic construct
CASSVTGGYGYTF

SEQ ID NO: 10 - protein, 10 aa, synthetic construct
FGQVGKILGE

SEQ ID NO: 11 - protein, 10 aa, synthetic construct
FGQIGKIRTT

SEQ ID NO: 12 - protein, 10 aa, synthetic construct
TAMIGKILGA

SEQ ID NO: 13 - protein, 10 aa, synthetic construct
FGQIGKIKGA

SEQ ID NO: 14 - protein, 10 aa, synthetic construct
FGQIGKILGA

SEQ ID NO: 15 - protein, 10 aa, synthetic construct
FGQIGTIKGA

SEQ ID NO: 16 - RNA, 20 nt, synthetic construct
  source 1..20: mol_type = other RNA
cggcccgcttctgctccagg

SEQ ID NO: 17 - RNA, 20 nt, synthetic construct
  source 1..20: mol_type = other RNA
cctggagcagaagcgggccg

(19) United States
(12) Patent Application Publication
(10) Pub. No.: US 2026/0250350 A1
(43) Pub. Date: Aug. 27, 2026

(54) ANCHOR-POSITION EDITING OF AN HLA ALLELE FOR TREATMENT OF AN AUTOIMMUNE DISEASE

(71) Applicant: THE REGENTS OF THE UNIVERSITY OF COLORADO, A BODY CORPORATE, Denver, CO (US)

(72) Inventors: Brian Freed, Aurora, CO (US); Christina Roark, Greenwood Village, CO (US)

(21) Appl. No.: 19/661,390

(22) Filed: Apr. 28, 2026

Related U.S. Application Data
(63) Continuation-in-part of application No. PCT/US25/51929, filed on Oct. 21, 2025.
(60) Provisional application No. 63/709,818, filed on Oct. 21, 2024.

(51) Int. Cl. C07K 14/705; A61K 35/28; A61K 38/00; A61P 37/06; C12N 9/22; C12N 15/11; C12N 15/86; C12N 15/90

(52) U.S. Cl. CPC C07K 14/70539; A61K 35/28; A61P 37/06; C12N 9/226; C12N 15/11; C12N 15/86; C12N 15/907; A61K 38/00; C12N 2310/20; C12N 2750/14143

(57) ABSTRACT

Methods are directed to modifying an HLA allele associated with an autoimmune disease. In one example, a method includes modifying an HLA allele such that the modified HLA allele encodes a non-polar amino acid instead of a target amino acid at an anchor position. A modified HLA protein encoded by the modified HLA allele can possess altered binding affinity for at least one self-peptide. In certain embodiments, the target amino acid is at anchor position 82 in a DRB1 or DQB1 allele or at position 80 in a DPB1 allele.

### CROSS REFERENCE TO RELATED APPLICATIONS

This application is a continuation-in-part of International Application No. PCT/US25/51929, on Oct. 21, 2025, titled “ANCHOR-POSITION EDITING OF AN HLA ALLELE FOR TREATMENT OF AN AUTOIMMUNE DISEASE,” which claims priority to and the benefit of U.S. Provisional Application No. 63/709,818, filed Oct. 21, 2024, titled “GENE EDITING OF ANCHOR AMINO ACIDS OF HLA ALLELES FOR TREATMENT OF AUTOIMMUNE DISEASE,” the disclosures of which are incorporated herein by reference in their entirety.

### TECHNICAL FIELD

The present disclosure relates to a recombinant HLA allele, a resulting protein, other compositions and methods for making such recombinant compositions, and methods of use of such recombinant compositions.

### SEQUENCE LISTING

The instant application contains a Sequence Listing which has been submitted electronically in XML format and is hereby incorporated by reference in its entirety. Said XML copy, created on Dec. 3, 2025, is named 11781300048.xml and is 15,989 bytes in size. The Sequence Listing does not extend beyond the scope of the specification, and does not contain new matter.

### BACKGROUND

Autoimmunity refers to a pathological condition in which the immune system generates an aberrant response against self-antigens, leading to the destruction or dysfunction of normal tissues. This phenomenon arises from a breakdown in the mechanisms of immune tolerance, which are designed to prevent the immune system from targeting the host's own cells. In autoimmune diseases, the immune system produces autoantibodies or autoreactive T cells that recognize and attack the body's own cells and tissues. The etiology of autoimmune disorders is multifactorial, involving genetic predispositions (such as specific HLA alleles), environmental triggers (such as infections, drugs, or stress), and potentially epigenetic modifications. Some autoimmune diseases such as rheumatoid arthritis (RA), Type 1 diabetes (TID), multiple sclerosis (MS), and sarcoidosis are prevalent and pose a substantial public health burden.

Human leukocyte antigen (HLA) refers to a group of related genes coding for proteins involved in immune function. HLA Class II are cell surface proteins responsible for antigen binding and presentation. Reference to alleles of the HLA proteins has a well-known nomenclature. For example, DRB1*01:01 refers to an allele of the DRB1 gene of the HLA complex. The value after the HLA gene designation and separated by * refers to the antigen group or level, and the value after the colon, refers to the allele variation in the peptide binding region at the protein sequence level. For example, DRB1*01:01 and DRB1*01:02 differ by two amino acids in the peptide binding region.

The main classes of HLA alleles include HLA Class I and HLA Class II alleles. HLA Class I present endogenous peptides (from proteins synthesized within the cell) to CD8⁺ T cells. They are expressed on almost all nucleated cells. The primary Class I genes are HLA-A, HLA-B, and HLA-C. HLA Class II present exogenous peptides (from proteins taken up by the cell) to CD4⁺ T cells. They are primarily expressed on antigen-presenting cells such as dendritic cells, macrophages, and B cells. The main Class II genes are HLA-DR, HLA-DQ, and HLA-DP. Each of these genes has multiple alleles, leading to a highly diverse set of HLA molecules. This diversity is crucial for the immune system's ability to recognize a wide range of antigens but also plays a significant role in transplant compatibility, autoimmune disease susceptibility, and vaccine responsiveness. HLA is the primary genetic factor related to autoimmune diseases, accounting for approximately half of known genetic predisposition. While over 200 associations between HLA and diseases have been described, deeper analysis and understanding of the role of HLA in treatment of autoimmune disease is still needed.

### SUMMARY

To address shortcomings in existing treatment and management of autoimmune disease, disclosed herein are methods for generating a recombinant HLA protein that is at least 95% identical to a wild-type (WT) HLA protein. In certain embodiments, the method includes a single amino acid substitution at an anchor position. The recombinant HLA proteins can be generated using an in vivo gene therapy approach, an ex vivo gene therapy approach, or a combination thereof.

Disclosed herein are methods and compositions useful in reducing autoimmunity in a subject suffering from or at risk of developing an autoimmune disease, disorder, or condition. Such diseases, disorders, or conditions include, without limitation, RA, TID, MS, sarcoidosis, celiac disease, Addison's disease, amyotrophic lateral sclerosis (ALS), Parkinson's disease, a delayed-type hypersensitivity reaction, asthma, autoimmune encephalitis, autoimmune pancreatitis, autoimmune thyroid disease, autoimmune uveoretinitis, Bechet's syndrome, Birdshot uveitis, Crohn's disease, dermatomyositis, eosinophilic granulamatosis with polyangiitis, glomerulonephritis, graft rejection, graft-versus-host disease, Graves' disease, Hashimoto's disease, Henoch-Schönlein purpura, idiopathic thrombocytopenia purpura, inflammatory bowel disease, interstitial lung disease, irritable bowel syndrome, Kawasaki disease, lupus nephritis, myasthenia gravis, myasthenic syndromes and neuromyelitis optica, myelin oligodendrocyte glycoprotein antibody disorders, narcolepsy, narcolepsy type 1, neuritis, vitiligo, paraneoplastic neurological syndromes, pemphigus vulgaris, pernicious anemia, polymyositis, primary biliary cirrhosis, psoriatic arthritis, pulmonary fibrosis or idiopathic pulmonary fibrosis, sarcoidosis, scleroderma, Sjögren's syndrome, systemic lupus erythematosus, T-cell mediated pulmonary disease, ulcerative colitis, and any other disease in which HLA class II genes are associated with the risk of developing the disease.

Certain HLA alleles, including alleles in the HLA-DRB1, HLA-DQB1, and HLA-DPB1 loci, have been associated with an increased risk of various autoimmune and other diseases. By way of example, HLA-DRB1 has been implicated in RA, MS, and TID; HLA-DQB1 has been implicated in TID, celiac disease, and narcolepsy; and HLA-DPB1 has been implicated in chronic beryllium disease and Graves' disease. The presence of such alleles does not guarantee disease development but may indicate a higher genetic predisposition. Environmental factors and other genetic components can also contribute to the pathogenesis of these conditions.

Provided here are methods for modifying an HLA allele associated with an autoimmune disease. One such method includes the steps for modifying an amino acid at an anchor position to another amino acid to modify the HLA allele, wherein a protein coded by the modified HLA allele possesses altered binding affinity for at least one self-peptide. An anchor position refers to a specific residue within the HLA protein that is critical for structural stability or binding interactions. In certain embodiments, the target amino acid at an anchor position 82 is asparagine in a DRB1 or DQB1 allele. In certain embodiments, the target amino acid at an anchor position 80 is asparagine in a DPB1 allele. The amino acid after editing at the anchor position is a non-polar amino acid, which results in the recombinant HLA protein having an antigen binding affinity different than antigen binding affinity of the wild-type HLA protein. The non-polar amino acid is one of alanine, valine, leucine, proline, methionine, tryptophan, glycine, isoleucine, or phenylalanine. The naturally occurring HLA proteins do not have a non-polar amino acid at this particular anchor position. For example, the DRB1*04:01^{N82L}, DRB1*04:01^{N82M}, DQB1*04:01^{N82L}, and DQB1*04:01^{N82M} proteins do not occur in nature.

Provided herein are methods of treating a subject suffering from or at risk of developing an autoimmune disease. One such method includes the steps for isolating hematopoietic stem and progenitor cells (HSPCs) from the subject and modifying the HSPCs to create modified HSPCs expressing a modified HLA allele.

Certain embodiments include a recombinant HLA protein comprising an amino acid sequence at least 95% identical to a wild-type HLA protein and an amino acid substitution at position 82 from asparagine to a non-polar amino acid. The recombinant HLA protein has an antigen binding affinity different than antigen binding affinity of the wild-type HLA protein and the non-polar amino acid is one of alanine, valine, leucine, proline, methionine, tryptophan, glycine, isoleucine, or phenylalanine. In certain embodiments, the non-polar amino acid is leucine. In certain embodiments, the non-polar amino acid is methionine. Embodiments also include a cell containing the recombinant HLA protein.

Certain embodiments include ex vivo methods of treating or preventing an HLA class II-associated autoimmune disease in a subject. One such method includes collecting and isolating the subject's hematopoietic stem and progenitor cells (HSPCs); editing the HLA allele in a genomic DNA of the HSPCs by replacing nucleotides encoding asparagine at amino acid residue position 82 with nucleotides encoding a non-polar amino acid; selecting, isolating, and optionally expanding the edited HSPCs; and administering the edited HSPCs into the patient, thereby treating or preventing the autoimmune disease in the subject. The non-polar amino acid can be alanine, valine leucine, proline, methionine, tryptophan, glycine, isoleucine, or phenylalanine.

Certain embodiments include in vivo methods of treating or preventing an HLA class II-associated autoimmune disease in a subject. One such method includes administering to a subject a delivery vehicle containing one or more of a nucleic acid, proteins, and/or a cofactor to effect editing of the HLA allele; editing the HLA allele in a genomic DNA by replacing nucleotides encoding asparagine at position 82 with nucleotides that encode a non-polar amino acid, thereby treating or preventing the autoimmune disease in the subject. The non-polar amino acid can be alanine, valine leucine, proline, methionine, tryptophan, glycine, isoleucine, or phenylalanine. Embodiments of the delivery vehicle include a viral vector, a lipid nanoparticle, or a virus-like particle. In certain embodiments, the delivery vehicle is a BactPac™ delivery vector.

Embodiments include gene-editing delivery vehicles for use in treating autoimmune diseases or conditions comprising a guide RNA sequence complementary to a target nucleic acid sequence within an HLA allele; a Cas9 protein or a similar endonuclease; and a template nucleic acid comprising a portion of the HLA allele with the nucleotides encoding asparagine at position 82 replaced with nucleotides encoding a non-polar amino acid. Embodiments of the delivery vehicle include a viral vector, a lipid nanoparticle, or a virus-like particle. In certain embodiments, the delivery vehicle is a BactPac™ delivery vector. In certain embodiments, the HLA allele is an HLA class II allele associated with an autoimmune disease.

Provided herein are methods of treating a subject suffering from or at risk of developing an autoimmune disease. One such method includes the steps for isolating CD34⁺ immune cells from the subject and modifying the CD34⁺ immune cells to create modified CD34⁺ immune cells expressing a modified HLA allele. The modified HLA allele encodes a protein with altered binding affinity for at least one self-peptide as compared to a protein coded by the wild-type HLA allele. Also disclosed herein are engineered HLA molecules having altered antigen binding and/or specificity compared to a non-engineered HLA molecule. In several embodiments, the antigen may be selected from various peptides including, without limitation, modified peptides, citrullinated peptides, hybrid peptides, and nucleic acids.

The variable region of an HLA molecule binds to specific peptide antigens, while the constant region has a more conserved structure. The high variability in the HLA variable region is due to numerous alleles that allow for the presentation of diverse peptides. The constant region has a more consistent function, like forming the structural backbone and binding to other immune cell receptors. As the constant region does not differ across alleles from autoimmune-susceptible and resistant subjects, this region has not been a target for therapeutic approaches. Asparagine at position 82 is present in all DRB1 and DQB1 alleles and is present at position 80 in DPB1 alleles. This asparagine residue provides one of 10-12 hydrogen bonding sites in the peptide binding groove. Therefore, it is a surprising result that editing this single amino acid had a significant effect on peptide binding. One would not seek to edit this amino acid at this position as asparagine is at position 82 in >99% of all Class II (DR, DQ and DP) alleles. Conventional approaches would focus on varying (polymorphic) amino acids in the peptide-binding region to study disease mechanisms, rather than editing a conserved or constant residue for therapeutic purposes. Allogeneic recognition and rejection also generally require that an HLA molecule be loaded with a peptide in the binding groove. In this context, the side chain of the peptide residue at position 82 is oriented upward out of the HLA groove (i.e., is “solvent exposed”), where it can contact the T cell receptor. Thus, during rejection, the T cell recognizes the composite surface formed by the foreign HLA molecule together with its bound peptide. Accordingly, reducing or eliminating peptide binding can diminish formation of this composite epitope and may reduce allogeneic T-cell recognition and rejection. Such an effect could have additional implications for bone marrow and solid organ transplantation.

The present disclosure is sufficient to enable one skilled in the art to practice the present disclosure. The present disclosure is not to be limited in scope by the constructs described, because the detailed embodiments are intended as illustrations of certain aspects of the present disclosure and any constructs that are functionally equivalent are within the scope of this disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. Embodiments are illustrated by way of example and not by way of limitation in the figures of the accompanying drawings.

FIG. 1A presents quantification of binding of native vimentin⁶⁶⁻⁷⁸ (left graph) and citrullinated vimentin⁶⁶⁻⁷⁸ (left graph) in resistant and susceptible alleles. FIGS. 1B and 1C are flow cytometric histograms of wild-type DRB1*04:01 and DRB1*04:01^{G86L} mutants binding to self-antigens (FIG. 1B) and vimentin peptides (FIG. 1C). FIG. 1D presents flow cytometric histograms of WT and DRB1*04:01^{G86F} mutants binding to vimentin peptides. The data for FIGS. 1B-1D are expressed as the mean fold increase of peptide binding to DRB1-transfected cells over binding to T2 controls.

FIG. 2A presents the protein structure of HLA-DRB1*04:01 bound to a peptide. FIG. 2B presents hydrogen bonds formed between the peptide and asparagine 82 of HLA-DRB1*04:01. The non-polar amino acid leucine is depicted next to asparagine. FIG. 2C presents molecular docking analysis showing N82 of DRB1*15:01 binds to peptides via hydrogen bonding. FIG. 2D presents molecular docking analysis showing position 82 of DQB1 forms hydrogen bonds with peptide backbone. FIG. 2E presents position 82 of DRB3 mediating hydrogen bonds with peptide backbone. FIGS. 2F and 2G presents the hydrophobicity maps of endogenous DRB1*04:01 protein and DRB1*04:01^{N82L} protein, respectively. FIG. 2H presents molecular docking of the DRB1*04:01^{N82L} mutant and shows no hydrogen bond formation between DRB1*04:01 and peptide. FIGS. 2I-2K present the hydrophobicity maps of binding of peptides by endogenous DRB1*04:01 protein, DRB1*04:01^{K71E} protein, and DRB1*04:01^{N82L} protein, respectively. FIGS. 2L-2N present the three approaches to HLA gene editing of DRB1*04:01 allele—polarization, pocket engineering, and amino acid editing at anchor positions, also known as anchor-position editing.

FIG. 3A presents the AAV repair construct to generate DRB1*04:01^{N82L} mutations, according to an embodiment of the disclosure. FIG. 3B presents an illustration of CRISPR/Cas9 targeting strategy to the DRB1*04:01 locus through gRNA mediated targeting, according to an embodiment of the disclosure. FIG. 3C presents the isolation, transduction, and transplantation protocol of human derived CD34⁺ stem cells, according to an embodiment of the disclosure.

FIG. 4A presents T-cell activation to DRB1*04:01 or DRB1*04:01^{N82L} in response to the collagen peptide. The graphs show the T-cell activation of four different T-cell hybridomas from DRB1*04:01⁺ rheumatoid arthritis patients. FIG. 4B presents flow cytometric histograms of HIP11 (a specific hybrid insulin peptide that is a potential autoantigen in TID) being assessed for its binding to Type-1 diabetes associated with DQ8 allele expressed in K562 cells. FIG. 4C presents graphical representation of T-cell activation in cells expressing wild-type DQB1*03:02 or DQB1*03:02^{N82L} proteins.

FIG. 5 presents binding of arthritogenic peptides—alpha enolase and vimentin—to the wild-type DRB1*04:01, and the edited DRB1*04:01^{N82L} and *04:01^{N82M} proteins.

FIG. 6 presents flow cytometric scatter plots and histograms of various peptides binding to endogenous DRB1*04:01 protein (top), DRB1*04:01^{N82L} protein (middle), or DRB1*04:01^{N82M} protein (bottom). The dot plot (left most column) is to show HLA-class II expression on the T2 cell line that were cloned to express respective HLA-II (endogenous, N82L, and N82M) as detected by the antibody WR18, which is a pan anti-HLA-class II antibody that stains and detects all HLA-class II alleles, such as DQ alleles associated with TID and DQ alleles associated with celiac disease, along with all DRB1 alleles associated with RA, MS and sarcoidosis.

FIG. 7A presents three-dimensional protein structures of the DRB1*04:01 protein. The relative locations of the modifications G86L, N82L, and K71E are indicated. FIG. 7B presents a ribbon model of the peptide binding region of the DRB1*04:01 peptide. It shows the location of the N82 residue in relation to the binding regions of a citrullinated peptide.

FIG. 8 presents a sequence alignment of DQB1*03:02 and DQB1*03:02^{N82L} alleles. The asparagine to leucine modification is indicated.

FIG. 9 presents the analysis of DQB1 peptide binding and T-cell activation gating strategy.

FIGS. 10A and 10B present dot plots, demonstrating that the N82L edit eliminates T-cell activation by HIP11.

FIG. 11 presents dot plots which show that the DQB1*03:02^{N82L} allele is expressed, and flow cytometric histograms that show that the N82L edit has limited binding of HIP11 at 100 μM in three separate cell samples.

FIG. 12A presents benefits of the BactPac™ gene delivery platform over legacy gene-editing delivery platforms. FIG. 12B presents a comparison of safety, toxicity, targeting specificity, delivery capacity, therapeutic window, and cost efficiency of BactPac™ over LNP, viral vectors, GalNAc delivery platforms. FIG. 12C presents the BactPac™ ligand targeting strategy, cell entry, and cargo release.

FIG. 13 presents flow cytometric histograms, which compare binding in untransfected, endogenous DRB1*15:01, and edited DRB1*15:01^{N82L} cells (right panels). Also shown is a ribbon model of the DRB1*15:01 protein and location of N82, which facilitates hydrogen bonding of ligand peptide molecules (left panel).

FIGS. 14A-14H present flow cytometric plots, which compare expression of HLA class II molecules on the plasma membrane of K562 cells transfected to express the indicated DQ2 and DQ8 molecules. Gating controls (unstained) are shown next to the stained cells (stained) and numbers indicate the % of live transfected cells that are positive for MHCII expression.

FIGS. 15A-15B present flow cytometric histograms, which compare binding in untransfected K562 cells (light histogram) and DQ8 (DQA1*03:01/DQB1*03:02) transfected cells (dark histograms).

FIGS. 16A and 16B present flow cytometric analysis of surface MHC expression in T2 lines expressing the alleles shown and peptide binding, respectively. FIG. 16B compares binding of an aquaporin 4 peptide by T2 cells expressing no HLA class II on the surface (light histogram in FIGS. 16B, top and bottom panels), DRB1*03:01 transfected cells with no edits (dark histograms in FIG. 16B, top panel), and DRB1*03:01 transfected cells with N82L edits (dark histograms in FIG. 16B, bottom panel).

### DETAILED DESCRIPTION

Disclosed herein are methods and compositions useful in reducing autoimmunity in a subject suffering from or at risk of developing an autoimmune disease, disorder, or condition. Such diseases, disorders, or conditions include, without limitation, RA, TID, MS, sarcoidosis, celiac disease, Addison's disease, amyotrophic lateral sclerosis (ALS), Parkinson's disease, a delayed-type hypersensitivity reaction, asthma, autoimmune encephalitis, autoimmune pancreatitis, autoimmune thyroid disease, autoimmune uveoretinitis, Bechet's syndrome, Birdshot uveitis, Crohn's disease, dermatomyositis, eosinophilic granulamatosis with polyangiitis, glomerulonephritis, graft rejection, graft-versus-host disease, Graves' disease, Hashimoto's disease, Henoch-Schönlein purpura, idiopathic thrombocytopenia purpura, inflammatory bowel disease, irritable bowel syndrome, Kawasaki disease, lupus nephritis, myasthenia gravis, myasthenic syndromes and neuromyelitis optica, myelin oligodendrocyte glycoprotein antibody disorders, narcolepsy, narcolepsy type 1, neuritis, vitiligo, paraneoplastic neurological syndromes, pemphigus vulgaris, pernicious anemia, polymyositis, primary biliary cirrhosis, psoriatic arthritis, pulmonary fibrosis or idiopathic pulmonary fibrosis, sarcoidosis, scleroderma, Sjögren's syndrome, systemic lupus erythematosus, T-cell mediated pulmonary disease, ulcerative colitis, and any other disease in which HLA class II genes are associated with the risk of developing the disease.

In particular embodiments according to the present disclosure, the disclosed autoimmune diseases are correlated with the presence of one or more HLA alleles. In most embodiments, HSPCs are mobilized and isolated from the subject, and the susceptible HLA allele is modified or replaced with an engineered HLA allele containing one or more amino acid substitutions of the target amino acid(s) at an anchor position of the protein coded for by the HLA allele. In certain embodiments, the amino acid is asparagine at position 82 in a DRB1 or a DQB1 allele, or at position 80 in a DPB1 allele.

Certain embodiments include a recombinant HLA protein comprising an amino acid sequence at least 95% identical to a wild-type HLA protein and an amino acid substitution at position 82 from asparagine to a non-polar amino acid. The recombinant HLA protein has an antigen binding affinity different than antigen binding affinity of the wild-type HLA protein and the non-polar amino acid is one of alanine, valine, leucine, proline, methionine, tryptophan, glycine, isoleucine, or phenylalanine. In certain embodiments, the non-polar amino acid is leucine. In certain embodiments, the non-polar amino acid is methionine. Embodiments also include a cell containing the recombinant HLA protein.

Position 82 is conserved as an asparagine residue in >99% of all Class II HLA (DR, DQ and DP) alleles. Asparagine at position 82 is common to all DRB1 and DQB1 alleles and presents at position 80 in DPB1 alleles. This asparagine provides one of about 10-12 hydrogen bonding sites in the peptide binding groove. Therefore, it is a surprising result that editing of this single amino acid had a significant effect on peptide binding. Its conservation across all Class II alleles indicates it may be critical for the structure and function. Conventional thinking in the art would be to modify the variable HLA amino acids to understand the role of the modifications in the treatment of disease, and not to edit the amino acids in the invariant or the conserved regions. In order for an allogeneic HLA molecule to be rejected, it still requires the presence of a peptide in the groove. With respect to the peptide, the side chain of the amino acid in position 82 faces upward out of the HLA groove (referred to chemically as “solvent exposed”) and interacts with the T cell receptor. The combination of peptide and foreign HLA molecule is what the T cell “sees” during rejection. Thus, reducing or eliminating peptide binding might also prevent rejection.

In certain embodiments, targeted engineering of an amino acid at an anchor position of the HLA gene modifies binding specificity and/or affinity to one or more self-antigens. The disclosed HLA mutations result in HLA protein changes that fail to trigger either rejection or graft-versus-host disease (GvHD) in a patient. In several embodiments, expression of the engineered HLA proteins on one or more antigen presenting cells in a subject suffering an autoimmune disease may result in amelioration of one or more symptoms associated with the autoimmune disease.

Also disclosed are methods of modifying a polar amino acid at an anchor position in a peptide binding region of an HLA allele. The method may further include substituting a polar amino acid with a non-polar amino acid, wherein the side chain of the non-polar amino acid can no longer form hydrogen bonds with the peptide present in the peptide binding region of an HLA allele. In various embodiments, the HLA allele may be selected from HLA-DRB1, HLA-DRB3, HLA-DRB4, HLA-DRB5, HLA-DQB1, and HLA-DPB1. Exemplary diseases and a non-exhaustive list of the strongly associated HLA alleles are shown in Table 1 below.

TABLE
Examples of diseases and the associated HLA alleles

| Disease | Strongly Associated HLA Alleles | Class |
|---|---|---|
| Type 1 Diabetes | DQB1*02:01, DQB1*03:02 | II |
| Celiac Disease | DQA1*05:01, DQB1*02:01, DQB1*03:02 | II |
| Narcolepsy (Type 1) | DQB1*06:02 | II |
| Multiple Sclerosis | DRB1*15:01 | II |
| Rheumatoid Arthritis | DRB1*01:01, DRB1*04:01, DRB1*04:04; | II |
| | DRB1*04:05, DRB1*10:01 | |
| Multiple Sclerosis | DRB1*15:01 | II |
| Neuromyelitis Optica | DRB1*03:01 | II |
| Sarcoidosis | DRB1*11:01 | II |

In these embodiments, the target amino acid may be, for example, position 82 and may be asparagine, serine, aspartic acid, glutamine, or histidine. The identity of the substituted amino acid may be valine, methionine, leucine, alanine, proline, glycine, isoleucine, tryptophan, and phenylalanine. In several embodiments, the HLA protein with the non-polar amino acid may possess lower binding affinity for at least one self-peptide associated with an autoimmune disease. In one aspect, Applicant provides methods to edit the edited HLA proteins, to prepare engineered HSPCs with edited HLA proteins, and also to treat autoimmune diseases using engineered autologous HSPCs with edited HLA proteins as presented in Table 2. The disclosed methods advantageously target specifically the underlying etiology of the patient's autoimmune disease while avoiding broad effects on other aspects of the patient's immune system. In the general population, individuals typically express approximately 10-16 HLA alleles, and at least about 90% of individuals express 10 or more HLA alleles. In contrast to pharmacologic agents that induce global or systemic immunosuppression, the methods and compositions described herein are designed to precisely edit a single HLA allele. These methods and compositions target an autoimmune disease at the source by intercepting self-reactive T-cell signaling, aiming to avoid the broad immunosuppression seen with current therapies. For example, these methods and compositions are positioned upstream in the disease pathway in contrast to existing treatment options for diseases that act by inhibiting specific components of the immune system that drive inflammation-including TNF inhibitors (Humira®, Enbrel®, Remicade®), IL-6R inhibitors (Actemra®, Kevzara®), anti-CD20 drugs (Rituxan®), CD80/86 inhibitors (Orencia®), and JAK inhibitors (Xeljanz®, Olumiant®, Rinvoq®).

TABLE 2
HLA Gene Editing for Autoimmune Diseases

| Disease | Alleles | Edits |
|---|---|---|
| Celiac Disease | DQB1*02:01, DQB1*03:02 | N82L, N82A |
| Type 1 Diabetes | DQB1*02:01, DQB1*03:02 | N82L, N82A |
| Rheumatoid Arthritis | DRB1*04:01, DRB1*04:04, | N82L, N82M |
| | DRB1*04:05, DRB1*01:01, | |
| | DRB1*10:01 | |
| Sarcoidosis | DRB1*11:01 | N82L |
| Multiple Sclerosis | DRB1*15:01 | N82L |
| Neuromyelitis Optica | DRB1*03:01 | N82L |

Certain embodiments include ex vivo methods of treating or preventing an HLA class II-associated autoimmune disease in a subject. One such method includes collecting and isolating the subject's hematopoietic stem and progenitor cells (HSPCs); editing the HLA allele in a genomic DNA of the HSPCs by replacing nucleotides encoding asparagine at amino acid residue position 82 with nucleotides encoding a non-polar amino acid; selecting, isolating, and optionally expanding the edited HSPCs; and administering the edited HSPCs into the patient, thereby treating or preventing the autoimmune disease in the subject. The non-polar amino acid can be alanine, valine leucine, proline, methionine, tryptophan, glycine, isoleucine, or phenylalanine.

Certain embodiments include in vivo methods of treating or preventing an HLA class II-associated autoimmune disease in a subject. One such method includes administering to a subject a delivery vehicle containing one or more of a nucleic acid, proteins, and/or cofactor to effect editing of the HLA allele; editing the HLA allele in a genomic DNA by replacing nucleotides encoding asparagine at position 82 with nucleotides that encode a non-polar amino acid, thereby treating or preventing the autoimmune disease in the subject. The non-polar amino acid can be alanine, valine leucine, proline, methionine, tryptophan, glycine, isoleucine, or phenylalanine. Embodiments of the delivery vehicle include a viral vector, a lipid nanoparticle, or a virus-like particle. In certain embodiments, the delivery vehicle is a BactPac™ delivery vector.

As used herein, the terms “treat,” “treating,” and “treatment,” and variations thereof, refer to alleviating, diminishing, arresting, abating, or ameliorating one or more symptoms of a disease or condition; preventing additional symptoms; ameliorating one or more underlying causes of symptoms; inhibiting the disease or condition (e.g., arresting development of the disease or condition); relieving the disease or condition; causing regression of the disease or condition; relieving a condition caused by the disease or condition; or stopping one or more symptoms of the disease or condition. “Treating” a disorder may refer to a reduction in severity of one or more symptoms associated with that disorder and does not necessarily require a complete reduction in severity of a symptom or a reduction of all symptoms and does not necessarily require complete resolution of the disorder.

Monocytes, macrophages, and dendritic cells (DCs) are the principal antigen-presenting cells that help to initiate and maintain the disease state in several autoimmune diseases. For example, in RA, the cells maintain the hyperinflammatory state in the joints associated with pain and debilitating progression of joint damage of the disease. However, these cells are short-lived and must be replenished from CD34⁺ HSPCs in the bone marrow. Monocytes, for example, typically survive in the blood for only a few days. However, if a monocyte migrates to an inflamed joint, they may progress to monocyte-derived DCs and macrophages and survive for weeks to months. Thus, the present disclosure describes replacing a subset of the cells in a patient's bone marrow with engineered HSPCs that will produce new engineered APCs that no longer present auto-immunogenic antigens, thus preventing activation of T cells and/or causing autoreactive T cells to revert to a quiescent memory state. In certain embodiments, the methods, compositions, and systems generally do not include depleting the patient's T cells and B cells prior to infusion of engineered HSPCs. Thus, these methods retain the patient's normal, innate, and adaptive immunity to infection by microbial pathogens and recognition of tumor or other antigens.

### Editing the HLA Allele for Expression by Engineered HSPC

Disclosed herein are methods for mutating amino acids for expression by engineered HSPCs. In several embodiments, the engineered HLA allele is expressed by engineered HSPCs of a patient to be treated by the disclosed therapy. In the case of RA, DRB1*04:04, DRB1*01:01, DRB1*04:05, DRB1*10:01, or DRB1*04:01 may be selected as an HLA allele for engineering. For example, DRB1*04:01 is seen in 31% of RA patients (compared to 10% of controls, p=10-3). The frequency of DRB1*04:01 increases with disease severity to greater than 50% of refractory or treatment-resistant RA patients and 88% of the most severe form of RA (Felty's Syndrome). In addition, DRB1*04:01 contributes the highest degree of susceptibility to RA.

In certain embodiments, the selected target amino acid position is at position 82. According to certain embodiments, HLA engineering is optimized to minimize or avoid entirely the consequences of HLA mismatching. Among recipients of allogeneic bone marrow transplants, any HLA disparity increases the risk of graft failure (rejection) and GVHD, so certain embodiments described herein include mutations within the peptide binding groove/cleft of the HLA molecule.

Described herein are methods for generating edited HLA alleles that are sufficient to alter antigen binding but do not elicit rejection. Specifically, HLA alleles that encode proteins with N82L and N82M mutations (or any non-polar amino acids at either positions 80 or 82) do not exist in nature.

Applicant demonstrated that mutating position 82 from a N to L substantially reduced the binding to autoimmune peptides. Using a peptide-binding assay, Applicant identified HLA allele DRB1*04:01 as possessing the greatest preference for a set of RA-associated antigens-including, post-translationally modified altered self-peptides. The collection of altered self-peptides includes a set of citrullinated peptide neoantigens that are upregulated during infection and inflammation. Human type II collagen is arthritogenic in animal models and in mice, CD4⁺ T cells that initiate arthritis recognize an immunodominant peptide located between amino acids 258-272 of collagen. CD4⁺ T cells that recognize the collagen²⁵⁸⁻²⁷² peptide are found in RA joints, and their presence in the peripheral blood at disease onset is associated with rapid progression of joint disease and poor responsiveness to conventional synthetic and biologic disease modifying antirheumatic drugs (DMARDs). The hydrogen bond between the polar N residue in position 82 and a residue in the collagen²⁵⁸⁻²⁷² peptide enhance peptide binding to DRB1*04:01. While a population of collagen-specific memory CD4+ T cells may persist in the patient, those cells may no longer receive the necessary TCR signals required to maintain chronic joint inflammation.

In certain embodiments, the engineered HSPCs engraft in the bone marrow within days and begin to generate DRB1*04:01^{N82L} expressing myeloid cells within ten days. In embodiments where the patients do not undergo immunosuppressive conditioning prior to administration of the disclosed engineered HSPCs, they will retain acquired T and B cell immunity present before the procedure. In embodiments where patients are treated with immunosuppressive conditioning, using low-dose melphalan or busulfan, and/or other agents, patients may experience a brief period of neutropenia and low platelet counts. These side effects should not be life-threatening and severe adverse events (SAEs) are not expected.

### Engineered HLA Molecules

Disclosed herein are various engineered HLA molecules. In some embodiments, the HLA molecule may be selected from one or more of HLA-DPA1, HLA-DPB1, HLA-DQA1, HLA-DQB1, HLA-DRB1, HLA-DRB3, HLA-DRB4, and HLA-DRB5.

In some embodiments, the HLA molecule may be selected from one or more of HLA-DRB1*01, HLA-DRB1*03, HLA-DRB1*04, HLA-DRB1*07, HLA-DRB1*08, HLA-DRB1*09, HLA-DRB1*11, HLA-DRB1*13, HLA-DRB1*15, and HLA-DRB1*16. In some embodiments, the HLA molecule may be selected from one or more of HLA-DQA1*01, HLA-DQA1*02, HLA-DQA1*03, HLA-DQA1*04, HLA-DQA1*05, HLA-DQA1*06, HLA-DQB1*02, HLA-DQB1*03, HLA-DQB1*04, HLA-DQB1*05, and HLA-DQB1*06.

Disclosed herein are various mutations at target amino acid positions within a mature HLA protein sequence. In several embodiments, the mutations are selected based on the criteria disclosed above. In some embodiments, specific allelic mutations may be selected based on the autoimmune disease or disorder to be treated.

The present disclosure includes methods of treating or preventing an autoimmune disease by administering engineered APCs and/or APC precursors, i.e., engineered HSPCs. In contrast to, for example, T cell therapies, the engineered cell compositions disclosed herein are provided to reduce or prevent T cell-mediated rejection responses, rather than elicit them. As such, the engineered compositions provide a relatively broad therapeutic window, while targeting the subject's specific condition. In certain embodiments, the methods comprise administration of a therapeutically effective amount of the engineered HSPCs. In certain embodiments, subjects receive 1×10⁶, 2×10⁶, 3×10⁶, 4×10⁶, 5×10⁶, for example 1-5 million, or more engineered, autologous, HSPCs per kg of body weight, such as by intravenous administration, in one or more doses, over one or more days.

The methods of the present disclosure include the production and administration of engineered HSPCs as described. In certain embodiments, the engineered HSPCs are autologous to the subject to be treated. Accordingly, some embodiments include isolating HSPCs or HSPC precursors from the subject, ex vivo engineering of the isolated HSPCs, optional selection and/or expansion of the engineered HSPCs, and administration of the engineered autologous HSPCs to the subject. Certain embodiments also include the mobilization and/or collection of the peripheral blood mononuclear cells (PBMCs) and/or bone marrow cells prior to the isolation of the HSPCs.

HSPCs or precursors can be collected (such as via apheresis) and isolated from subjects by methods known in the art. For example, PBMCs and/or bone marrow cells can be mobilized, collected (such as via apheresis), isolated, and HSPCs purified based on expression of CD34. HSPC subpopulations can be selected based on expression of additional antigens as desired. Additionally, or alternatively, HSPCs can be produced from precursor cells, such as pre-harvested stem cells or de-differentiated cells of the subject, as known in the art. Although autologous HSPCs are presently certain, the present disclosure is not limited to autologous HSPCs. For example, in certain embodiments, non-autologous (donor) HSPCs engineered to express a desired HLA without expressing proteins capable of eliciting non-self-responses are provided.

Certain embodiments of the methods provided herein also include pre-conditioning, such as non-myeloablative conditioning, and/or post-treatment interventions, such as to promote engraftment of the engineered HSPCs. Additionally, or alternatively, HSPC engineering according to the present disclosure can be performed in vivo, such as by administration of viral vectors encoding, inter alia, expressed autoimmunity resistance alleles and/or gene-editing constructs as described herein. Moreover, although reference is primarily made herein to single engineered HSPC populations, multiple HSPC compositions having discrete HLA allelic modifications may also be provided, separately or sequentially, such as in instances of multi-allelic autoimmune disease.

### HLA Allele Engineering

The present disclosure includes systems, constructs, and techniques for gene editing, and the application of same to provide resistance to autoimmunity. In particular, certain embodiments of the present disclosure include constructs, systems, and vectors for HLA allele engineering as disclosed herein.

Several gene-editing systems are available, suitable, and well-characterized in the art. For example, in certain embodiments, CRISPR-Cas systems containing a DNA targeting polynucleotide complementing the HLA allele to be modified and a CRISPR-associated nuclease, such as Cas9, are provided. Related CRISPR-Cas9 systems for treatment of RNA are disclosed in PCT/US2018/029302, published as WO2018200635, hereby incorporated by reference herein in its entirety.

In other embodiments, CRISPR systems containing, for example, CasX, Cas12a, Cas13, or MAD7 are provided for HSPC HLA allele engineering, for example as in PCT/US2019/043066, published as WO/2020/023529, and PCT/US2018/028919, published as WO/2018/195545. Certain CRISPR systems can be selected on the basis of protospacer-adjacent motif (PAM) specificity, allowing targeting of almost all genomic sequences, on-target selectivity, efficiency in human HSPCs, and other considerations. In alternative embodiments, TAL effector nucleases (TALENs) or zinc finger nucleases (ZFN s) are employed for HLA allele engineering as disclosed at Nucleic Acid Res. 2011 Sep. 1; 39 (17):7879).

In further embodiments, HLA allele engineering is performed with fusion proteins, such as enzymatically inactive dCas9-based fusion proteins. These systems combine the programmable DNA-targeting capability associated with CRISPR with additional on-target selectivity and/or functional capabilities of other gene engineering platforms. For example, in certain embodiments, HLA allele engineering is performed with systems including a Cas-CLOVER fusion, as described in PCT/US2015/036226.

In additional embodiments, including certain embodiments, HLA allele engineering is performed with a nucleobase editing system. For example, certain embodiments provide an HLA-allele targeting polynucleotide and a fusion protein comprising dCas9 and a nucleobase editing enzyme, such as a deaminase. Such embodiments advantageously result in generation of specific point mutations sufficient to alter the amino acid encoded at the targeted HLA allele codon without resulting in or requiring DNA double-strand breakage and repair.

The principles of design for CRISPR-Cas systems and vectors for same are well known in the art, and in the present context essentially require only selection of a sequence complementary to the portion of the HLA allele to be engineered. The same is true with respect to CRISPR-Cas fusion-based systems including the examples described. The production of genetic engineering platforms involving protein-based DNA targeting, such as TALENs and zinc fingers, is also well-characterized, and suitable such systems for use with the present disclosure can be generated with no more than routine procedures and experimentation.

In additional and alternative embodiments, the HLA allele engineering systems include a homologous repair template. For example, in certain embodiments, the entire gene for the susceptible HLA allele, within the MHC locus, can be excised and replaced with the engineered HLA allele. In several embodiments, the gene coding for the susceptible HLA allele may be disrupted by insertion of the engineered HLA allele, which may be as an uninterrupted nucleic acid with the engineered HLA allele's cDNA sequence.

HLA allele engineering, and the systems therefore, according to the present disclosure can also include, for example, vectors, such as retroviral vectors for expression of the HLA allele engineering constructs disclosed. Transient transfection techniques and systems therefore can also be applied. Accordingly, the present disclosure is not limited by or to specific HLA allele engineering constructs or systems.

### Engineered Hematopoietic Stem and Progenitor Cells

Autologous immune cells may be engineered using various systems as disclosed herein, for example cells may be engineered to carry and express engineered HLA genes and molecules with various viral vectors and/or nucleases capable of genomic editing. Various protocols well known to those of skill in the art may allow for screening of the genomes of manipulated cells to assess the frequency and/or position of viral insertions, double strand breaks in DNA (DSBs) or other potentially mutagenic events (Li H, Haurigot V, Doyon Y, et al. In vivo genome editing restores haemostasis in a mouse model of haemophilia. Nature. 475(7355):217-21, 2011). In several embodiments, the systems may be useful in removing and or preventing expression of the susceptible HLA allele, as well as inserting the engineered HLA allele into the same locus. In several embodiments, the engineered HLA allele is expressed from a cDNA sequence.

In certain embodiments, autologous immune cells, including HSPCs can be engineered directly in vivo. Embodiments include gene-editing delivery vehicles for use in treating autoimmune diseases or conditions comprising a guide RNA sequence complementary to a target nucleic acid sequence within an HLA allele; a Cas9 protein or a similar endonuclease; and a template nucleic acid comprising a portion of the HLA allele with the nucleotides encoding asparagine at position 82 replaced with nucleotides encoding a non-polar amino acid. In certain embodiments, the HLA allele is an HLA class II allele associated with an autoimmune disease. For example, gene-editing constructs can be delivered directly to the subject. Delivery vehicles for in vivo gene editing include viral vectors (such as adeno-associated viruses, lentivirus, and adenovirus), non-viral vectors (such as lipid nanoparticles, cell-penetrating peptides, virus-like particles, enveloped delivery vehicles, and nanoparticles), and physical methods (such as microinjection, electroporation, and hydrodynamic delivery). Embodiments of the delivery vehicle include a viral vector, a lipid nanoparticle, or a virus-like particle. In certain embodiments, the delivery vehicle is a BactPac™ delivery vector.

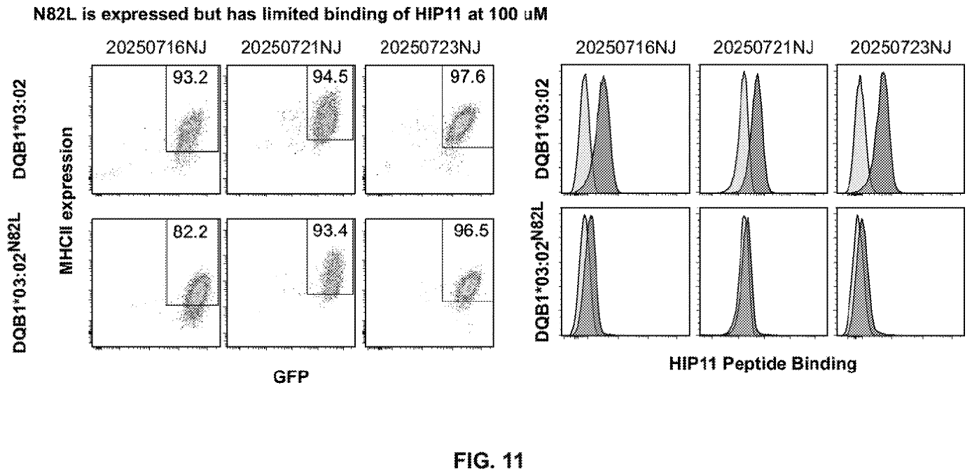

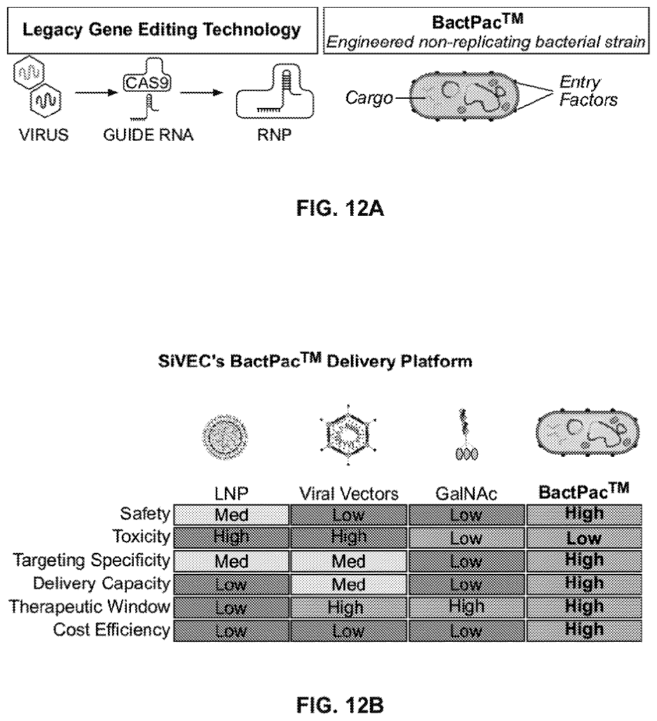

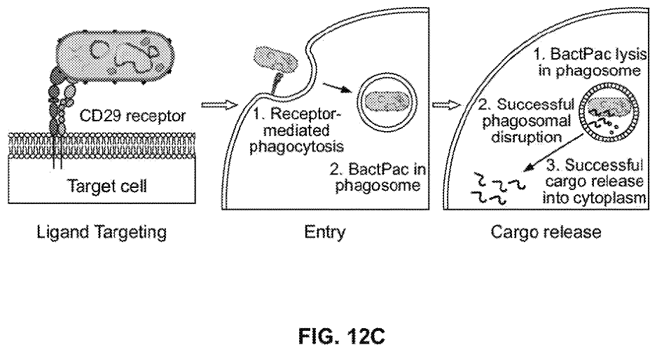

In an embodiment, gene-editing constructs can be delivered by implementing one of SiVEC's BactPac™ delivery vehicle (see, FIGS. 12A-12C), as described in patent application nos. PCT/US2018/025961, PCT/US2020/035613, PCT/US2021/012992, PCT/US2022/031949, and PCT/US2023/019838. BactPac™ is a non-immunogenic, non-replicating, and non-integrative bacterial vehicle that can produce and deliver the entire gene-editing cargo. The constructs may be delivered directly to the patient such that the HSPCs of a subject are modified in vivo. It uses normal flora bacteria (e.g., E. coli) that are engineered to (1) produce therapeutic nucleic acids, proteins, and gene editors, (2) deliver these therapeutic payloads to targeted tissues, and (3) release the payload into the targeted cell. BactPac™ cells can be engineered to express a single domain antibody to target the delivery vehicle to a specific cell type (see, FIG. 12C). This delivery vehicle provides for precise delivery of the CRISPR-based genome editing machinery to CD34⁺ HSPCs in a subject. The vehicles can deliver Cas9 or other endonuclease, single-guide RNA (sgRNA), and a repair template to facilitate editing and homology directed repair (HDR) for the HLA gene. The BactPac™ strain can be engineered for intracellular delivery of gene-editing components to target and edit the human HLA locus BB1*04:01 allele using the strain titled “BactPac-RG0401.” BactPac™ can also be employed ex vivo.

Therapeutically relevant levels of genetically modified engineered HSPCs needed to effect clinical outcomes may be more readily achieved by expansion of large populations of cells ex vivo and reintroduction(s) into the patient.

### EXAMPLES

### Example 1: DRB1*04:01 Pocket 1 Mutants Still Bind Citrullinated Vimentin Peptides

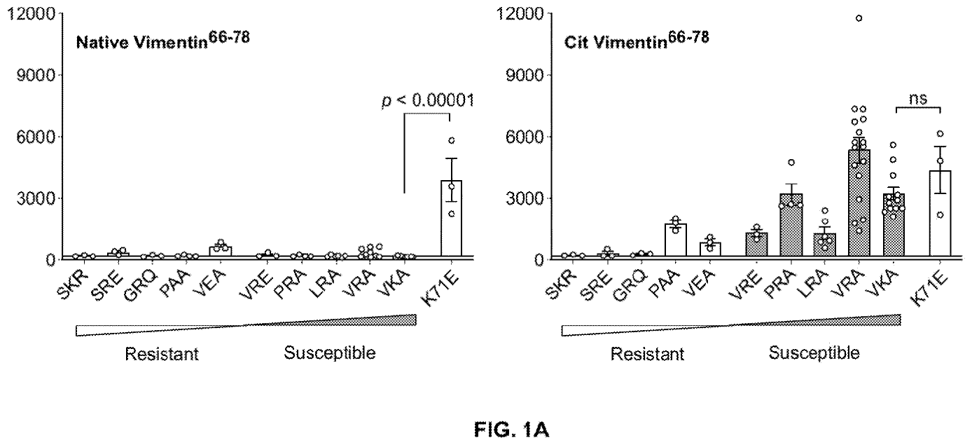

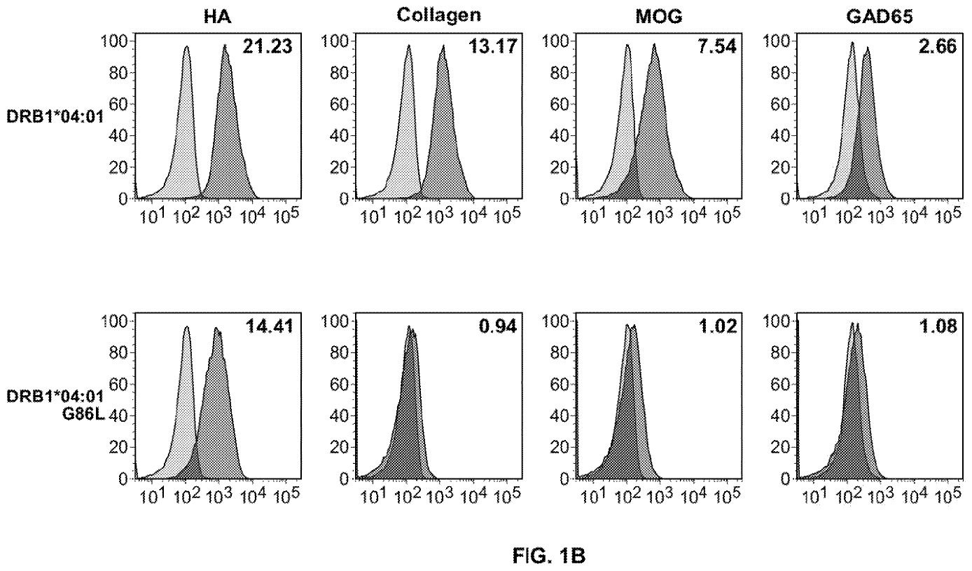

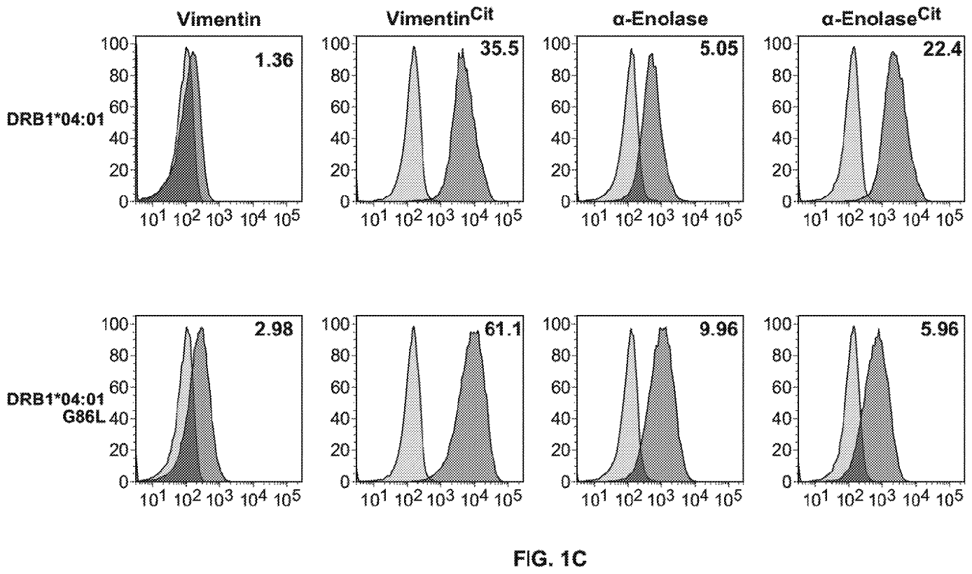

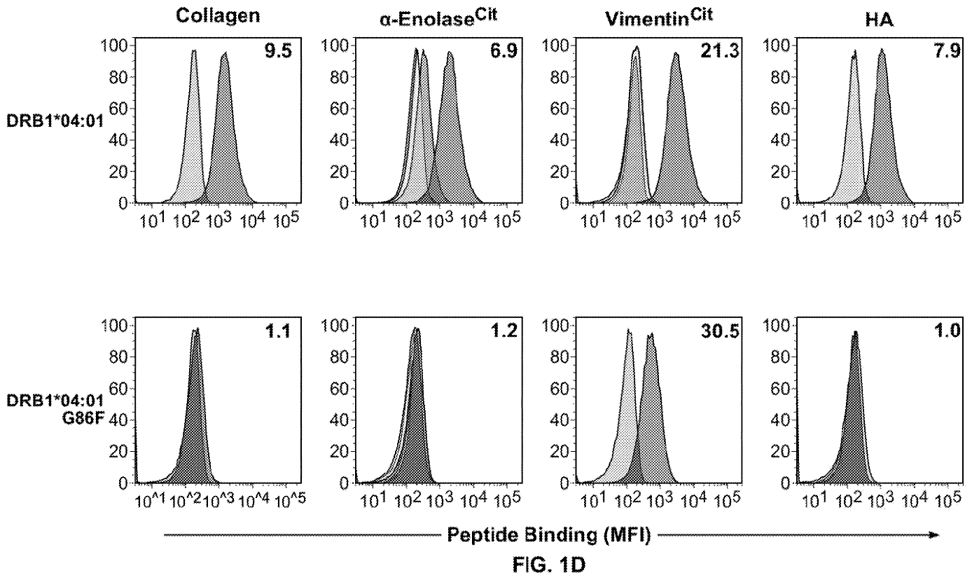

The ability of DRB1*04:01^{K71E} to bind native and citrullinated peptides was measured compared to both resistant and susceptible HLA alleles. FIG. 1A presents quantification of binding of native vimentin⁶⁶⁻⁷⁸ (left graph) and citrullinated vimentin⁶⁶⁻⁷⁸ (left graph) in resistant and susceptible alleles. FIGS. 1B and 1C are flow cytometric histograms of wild-type DRB1*04:01 and DRB1*04:01^{G86L} mutants binding to self-antigens (FIG. 1B) and vimentin peptides (FIG. 1C). FIG. 1D presents flow cytometric histograms of WT and DRB1*04:01^{G86F} mutants binding to vimentin peptides. The data for FIGS. 1B-1D are expressed as the mean fold increase of peptide binding to DRB1-transfected cells over binding to T2 controls.

As depicted in FIG. 1, the DRB1*04:01^{K71E} binds to native vimentin peptide stronger than endogenous susceptible alleles (left graph). Additionally, the DRB1*04:01^{K71E} still shows propensity for binding citrullinated vimentin peptide to a similar degree as the susceptible alleles (right graph). These data indicate the DRB1*04:01^{K71E} may not confer autoimmunity resistance in diseases that are mediated via vimentin binding to HLA. Other amino acids within pocket 1 of the antigen binding cleft were mutated to determine the binding capacity to self-peptides and citrullinated vimentin peptides. As shown in FIG. 1B, DRB1*04:01^{G86L} mutants do not bind to the self-antigens, such as collagen, MOG, and GAD65. However, the DRB1*04:01^{G86L} mutant still binds to citrullinated vimentin peptides as shown in FIG. 1C. Additional mutants were generated to generate G86 phenylalanine mutants. Similar to the DRB1*04:01^{G86L} mutants, the DRB1*04:01^{G86F} mutants still displayed binding capacity for citrullinated vimentin peptides (FIG. 1D). These data indicate targeting pocket 1 of HLA DRB1*04:01 can hinder binding to self-antigen, it does not inhibit binding to citrullinated vimentin peptides.

### Example 2: Asparagine 82 in Endogenous HLA-DRB1 Mediates Peptide Binding

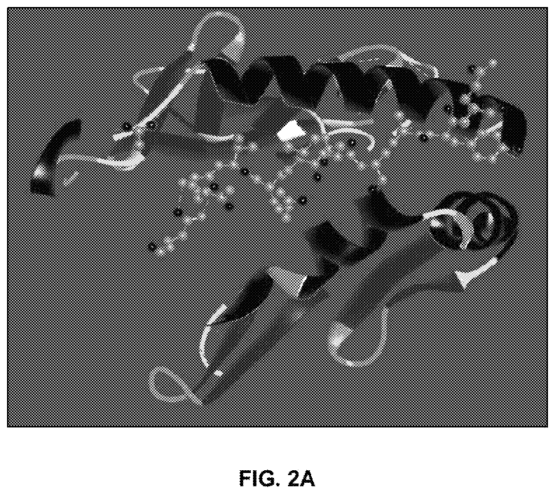

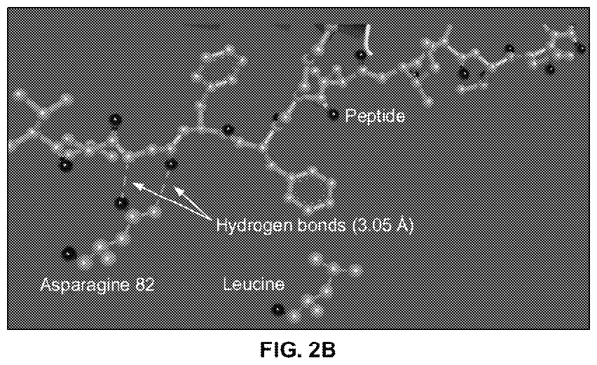

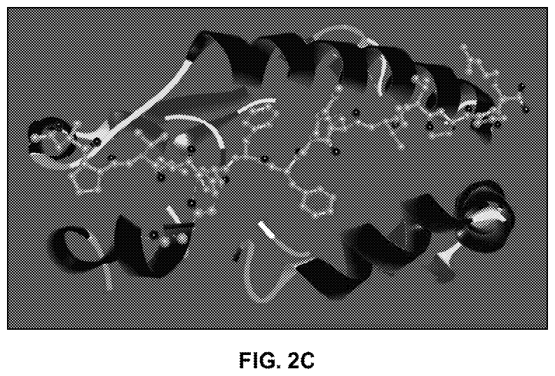

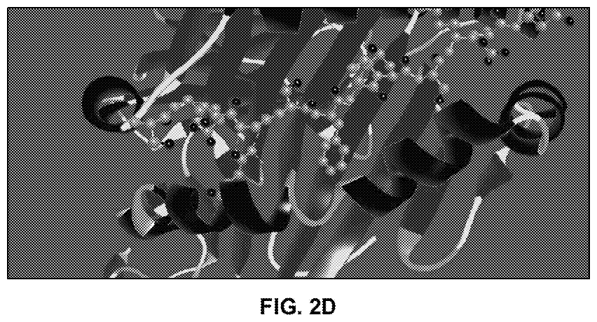

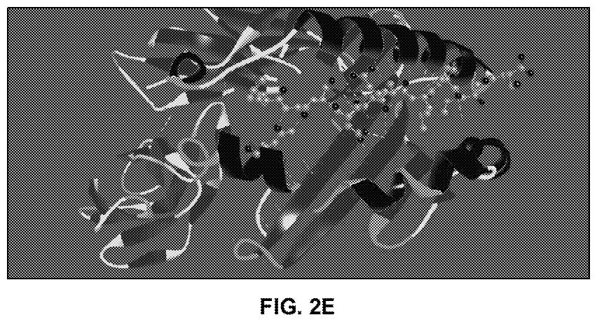

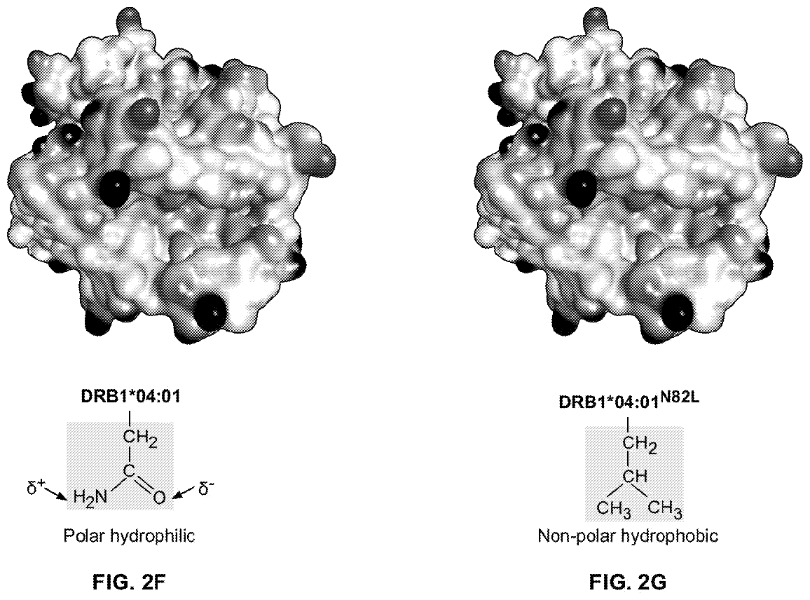

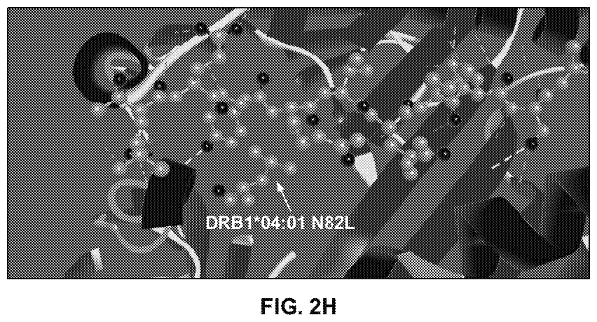

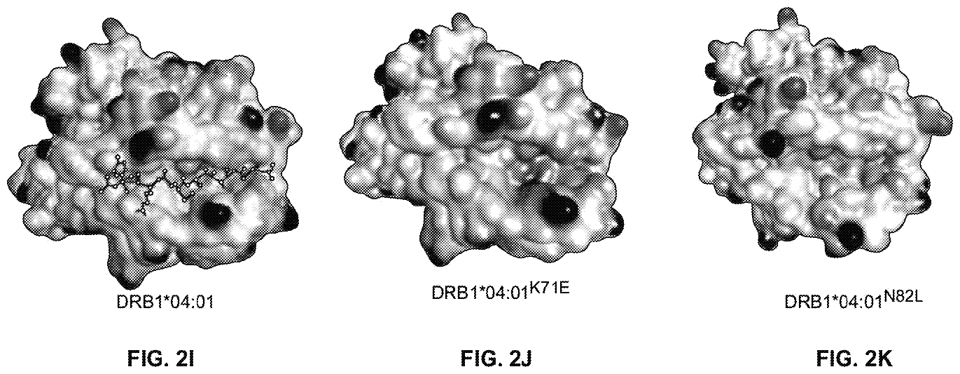

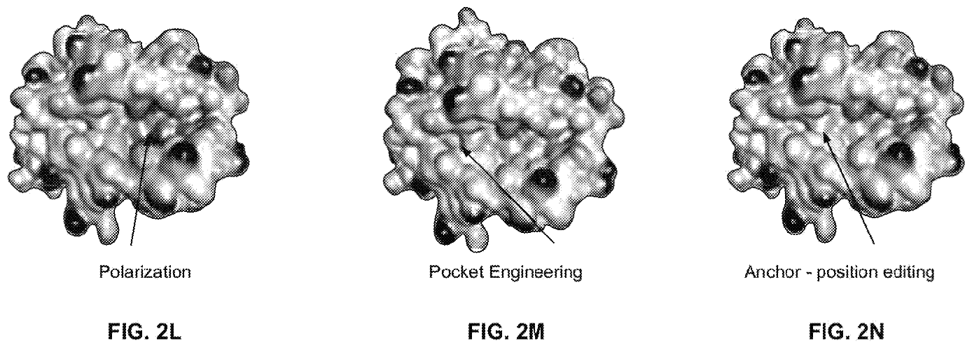

FIG. 2A schematically presents the protein structure of the HLA-DRB1 protein in the context of peptide binding. As outlined in FIG. 2B, the polar hydrophilic amino acid asparagine at position 82 within the antigen binding cleft mediates hydrogen bonds with cognate peptides. Additionally, position 82 in DRB1*15:01 (FIG. 2C), DQB1 (FIG. 2D), and DRB3 (FIG. 2E) mediates hydrogen bonding between HLA and cognate peptides. Therefore, an HLA protein wherein asparagine 82 was replaced with a non-polar hydrophobic amino acid was generated. The resulting mutant HLA protein contains at least 95% sequence identity with wild-type HLA but harbors a hydrophobic amino acid such as leucine at position 82, which subsequently alters the hydrophobicity of the antigen binding pocket (FIGS. 2F and 2G). Additionally, molecular docking analysis reveals DRB1*04:01^{N82L} mutants do not form hydrogen bonds with peptides (FIG. 2H). FIGS. 2I-2K present representations of the effects on binding of peptides following HLA gene editing of DRB1*04:01 allele. The wild type DRB1*04:01 protein (FIG. 2I) has a strong preference for citrullinated peptides and collagen. The edited DRB1*04:01^{K71E} protein (FIG. 2J) does not bind collagen, acquires binding to native vimentin, and still binds to citrullinated vimentin. The edited DRB1*04:01^{N82L} protein (FIG. 2K) does not bind collagen or the citrullinated peptides. And, this change in binding profile is applicable to proteins resulting from editing of all DRB1 and DQB1 alleles.

FIGS. 2L-2N present three innovative approaches to HLA gene editing of DRB1*04:01 allele-polarization, pocket engineering, and anchor-position editing. The polarization approach (FIG. 2L) results in tolerance to collagen with a minimal degree of allogenicity. However, the polarization approach does not reduce binding to citrullinated peptides and does not generally reduce an allele's peptide-binding repertoire; for example, it increases peptide binding to native vimentin. The polarization approach has been described in PCT patent application No. PCT/US2018/029302, which describes RNA-guided genome editing to introduce a point mutation in the DRB1*04:01 allele (e.g., K71E) in hematopoietic stem and progenitor cells (HSPCs), followed by autologous transplantation of the edited cells to bias immune responses toward a tolerant phenotype. The pocket engineering approach (FIG. 2M) results in tolerance to collagen and occlusion of Pocket 1. But, pocket engineering still resulted in the mutant proteins with binding capacity for one or more citrullinated peptides and increased binding to certain self-peptides. The pocket engineering approach has been described in PCT patent application Nos. PCT/US2022/028644, PCT/US2022/028643, and PCT/US2022/028645, which describe engineering one or more amino acid substitutions within the HLA antigen-binding cleft (including residues forming Pocket 1) to remodel pocket geometry, thereby reducing binding of selected autoantigenic peptides while retaining overall HLA expression and function, and delivering such engineered HLA alleles via edited autologous hematopoietic stem cells. The anchor-position editing approach (FIG. 2N) results in tolerance to collagen and loss of binding of both native vimentin and citrullinated peptides as this edit generally reduces an allele's peptide-binding repertoire. Notably, the results presented herein demonstrate that a single substitution at the conserved anchor residue (e.g., N82L) can abolish hydrogen-bonding interactions that broadly are required for stable peptide loading, thereby producing a global dampening of peptide presentation rather than a peptide-by-peptide redesign. In contrast to polarization and pocket-focused strategies that can preserve or even enhance binding of certain self-peptides, anchor-position editing is shown to eliminate binding to multiple disease-relevant antigens and, in functional assays, to eliminate T-cell activation driven by those peptides. Accordingly, anchor-position editing provides a mechanistically distinct and broadly applicable route to reduce autoimmune triggering across multiple DRB1, DQB1, and other HLA Class II alleles while still permitting residual binding to selected protective peptides, as exemplified by the retained low-level hemagglutinin binding observed for N82L.

### Example 3: Generation of HLA-DRB1*04:01^{N82L} Mutations

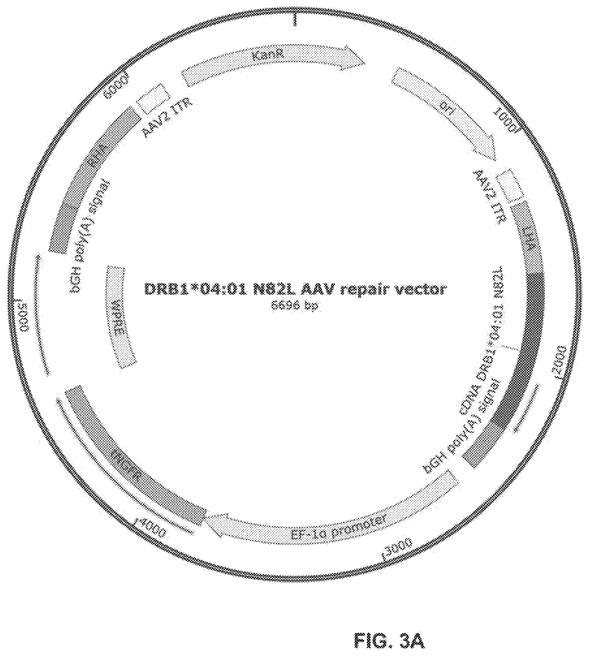

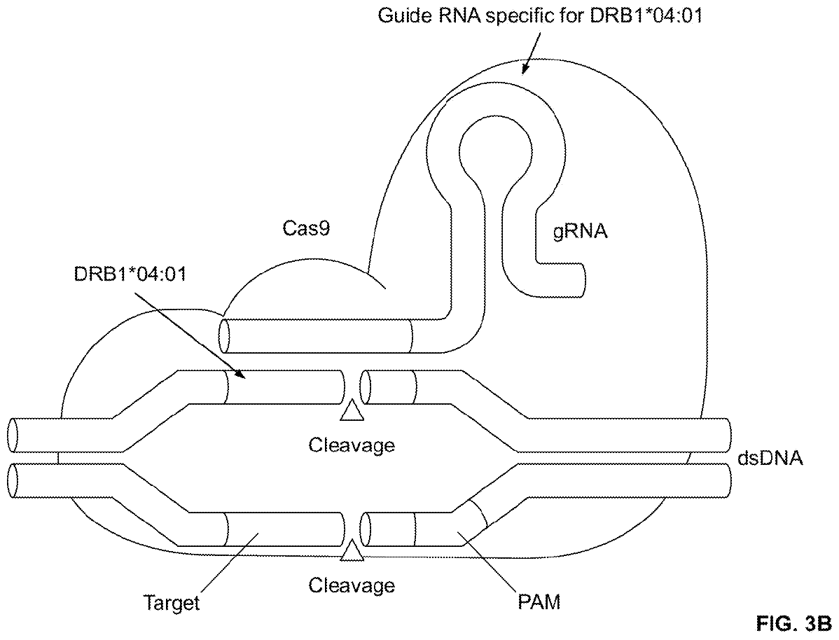

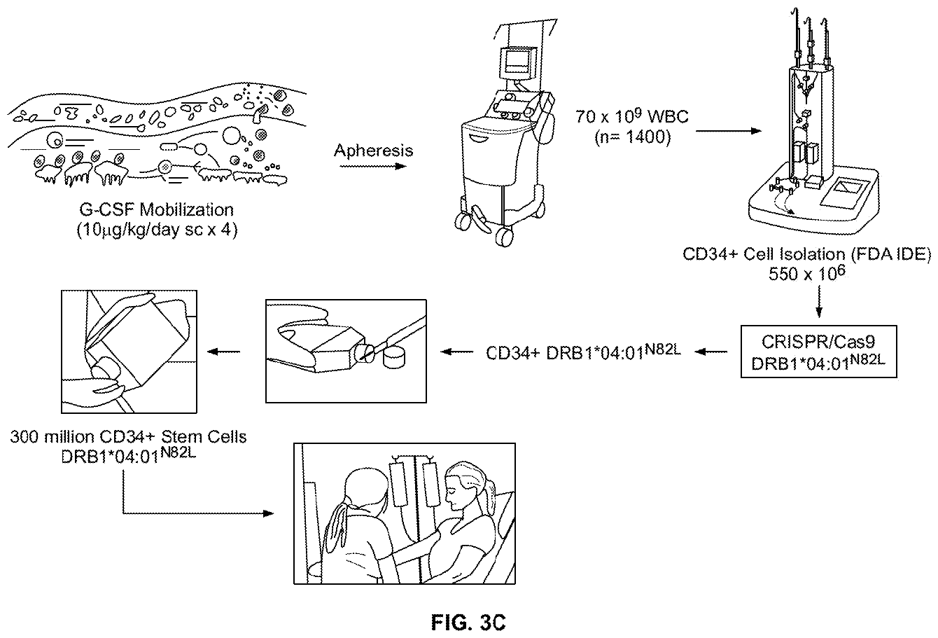

Single amino acid substitution of asparagine 82 with leucine mutants are generated. FIG. 3A presents the AAV repair construct to generate DRB1*04:01^{N82L} mutations, according to an embodiment of the disclosure. FIG. 3B presents an illustration of CRISPR/Cas9 targeting strategy to the DRB1*04:01 locus through gRNA mediated targeting, according to an embodiment of the disclosure. FIG. 3C presents the collection, isolation, transduction, and transplantation protocol of human derived CD34⁺ stem cells, according to an embodiment of the disclosure.

Briefly, an adeno-associated viral (AAV) repair vector is engineered containing the cDNA sequence for codons 22-90 of the DRB1*04:01 locus (FIG. 3A). The endogenous asparagine codon (AAC) was replaced with a leucine codon (CTA) in the cDNA sequence to achieve single amino acid substitution. Single amino acid substitution is performed via CRISPR/Cas9 mediated cleavage. Specifically, guide RNAs (gRNAs) designed to the DRB1*04:01 locus are used to direct Cas9 mediated cleavage of the target sequence (FIG. 3B). In certain embodiments, the guide RNA sequences that specifically target DRB1*04:01 are 185/rev (5′cggcccgcttctgctccagg 3′) and 208/fwd (5′cctggagcagaagcgggccg 3′).

The AAV vectors containing the asparagine to leucine substitution are used to transduce human CD34+ stem cells (FIG. 3C). Briefly, patients are stimulated for four days subcutaneously with 10 μg/kg with granulocyte colony-stimulating factor (G-CSF) to mobilize white blood cells (WBCs). 70×10⁹ WBCs are subject to CD34⁺ selection. The subsequent isolated CD34⁺ stem cells are transduced with CRISPR/Cas9 constructs to generate CD34⁺ DRB1*4:01^{N82L} mutant cells. Three hundred million CD34⁺ DRB1*04:01^{N82L} mutant cells are infused once leading to rapid engraftment (10 days) without the need for long-term immunosuppression subsequent to the administration of the engineered cells.

### Example 4: Editing Asparagine to a Non-Polar Amino Acid Residue at Position 82 of DRB1*04:01 Blocks Nearly all Peptide Binding and T-Cell Activation

To evaluate the effects of mutating asparagine at position 82 to leucine on peptide binding, endogenous DRB1*04:01 and DRB1*04:01^{N82L} mutants were incubated with a variety of biotinylated peptides known to bind to HLA-DRB1. Unbound peptides were washed followed by incubation with PE-conjugated streptavidin beads. Flow cytometry was subsequently performed to determine peptide binding. Incubation of endogenous DRB1*04:01 with the peptides led to binding in all cases except native vimentin. On the contrary, incubation of the DRB1*04:01^{N82L} mutant proteins blocked the binding of collagen, citrullinated α-enolase, and citrullinated vimentin.

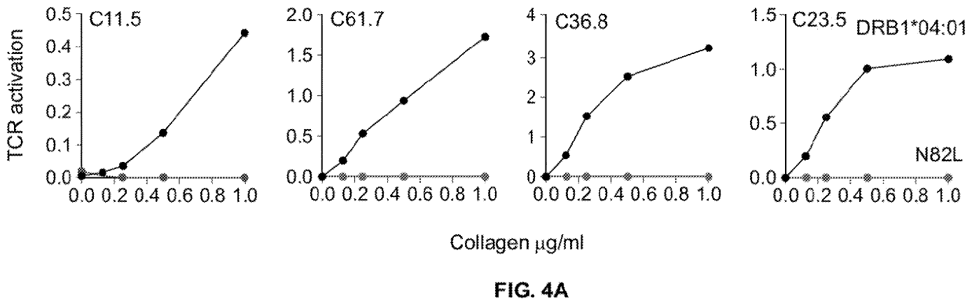

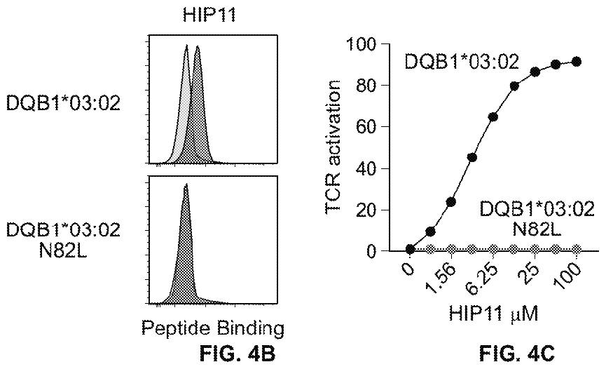

FIG. 4A presents dose response curves that show T-cell activation in response to increasing collagen concentrations for 4 different T-cell hybridomas derived from RA patients. Highly active T-cell responses to collagen presented by DRB1*04:01 is shown whereas there is no measurable T-cell response generated from collagen presented by the edited DRB1*04:01^{N82L} cells.

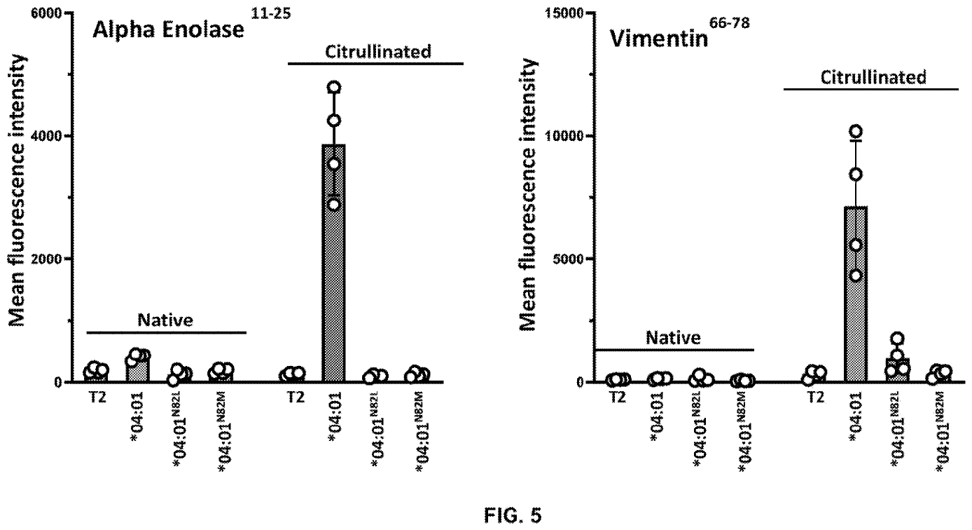

FIG. 5 presents binding of arthritogenic peptides—alpha enolase and vimentin—to position 82 (pocket 1) edits of DRB1*04:01, DRB1*04:01^{N82L} and DRB1*04:01^{N82M}. Native alpha enolase and vimentin show little to no binding, citrullinated alpha enolase shows high levels of binding to the DRB1*04.01 allele, which binding is ablated by both N82L and N82M edits.

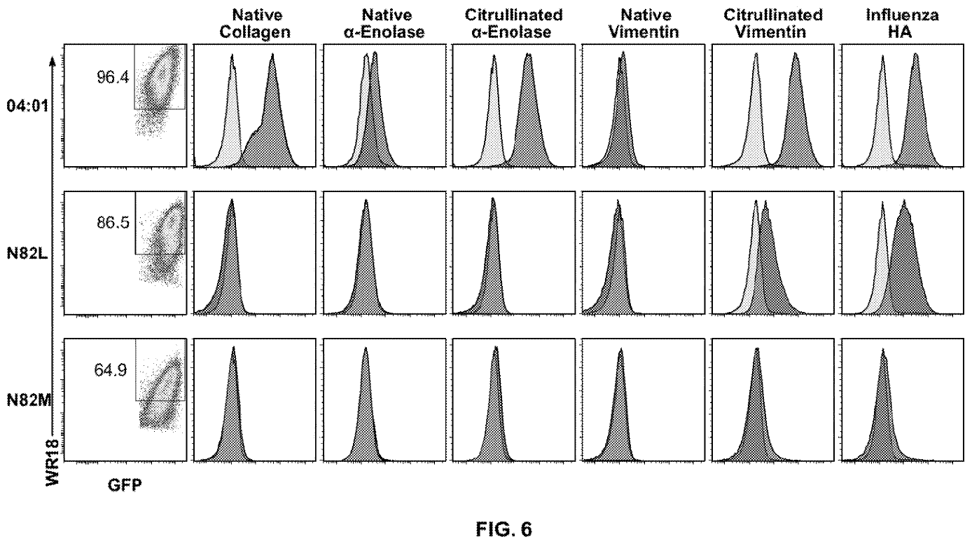

FIG. 6 presents flow cytometric scatter plots and histograms of various peptides binding to endogenous DRB1*04:01 protein (top), DRB1*04:01^{N82L} protein (middle), or DRB1*04:01^{N82M} protein (bottom). The dot plot (left most column) is to show HLA-class II expression on the T2 cell line that were cloned to express respective HLA-II (endogenous, N82L, and N82M) as detected by the antibody WR18, which is a pan anti-HLA-class II antibody that stains and detects all HLA-class II alleles, such as DQ alleles associated with TID and DQ alleles associated with celiac disease, along with all DRB1 alleles associated with RA, MS and sarcoidosis.

As depicted in FIG. 6 (top panels), incubation of endogenous DRB1*04:01 with the peptides led to binding in all cases except native vimentin, as evidenced by the right shift in the top histograms (dark gray peaks) compared to the isotype controls (light gray peaks). The bottom two sets of panels show that incubation of the DRB1*04:01^{N82L} and, separately, of the DRB1*04:01^{N82M} mutant proteins blocked the binding of collagen, citrullinated α-enolase, and citrullinated vimentin (bottom panels), as evidenced by the overlapping peaks. Methionine is a larger amino acid than leucine and thus has a higher likelihood of disrupting HLA 3-dimensional structure. As shown in the histograms, the N82M edit results in complete loss of HA binding, whereas N82L still retains low binding to HA, which can be advantageous for retaining protection against infectious agents. There was a 100% reduction of TCR activation by the DRB1*04:01^{N82L} edits. The quantified results are summarized in Table 3 below:

TABLE 3
Peptide Binding for DRB1*04:01 allele N82L and N82M Edits

| | | Peptide Binding Ratio | | Peptide |
|---|---|---|---|---|
| Alleles | | (values over 1 are positive) | | Binding |
| and Edits | Peptide | Unedited | Edited | % Reduction |
| DRB1*04:01 | Collagen | 46 | 1 | 100% |
| N82L | Native alpha-Enolase | 2 | 1 | 99% |
| | Citrullinated alpha-Enolase | 33 | 1 | 100% |
| | Native Vimentin | 1 | 1 | 100% |
| | Citrullinated Vimentin | 25 | 3 | 93% |
| | Hemagglutinin (Influenza A) | 12 | 5 | 62% |
| DRB1*04:01 | Collagen | 46 | 1 | 100% |
| N82M | Native alpha-Enolase | 2 | 1 | 96% |
| | Citrullinated alpha-Enolase | 33 | 1 | 100% |
| | Native Vimentin | 1 | 1 | 100% |
| | Citrullinated Vimentin | 25 | 1 | 100% |
| | Hemagglutinin (Influenza A) | 12 | 1 | 100% |

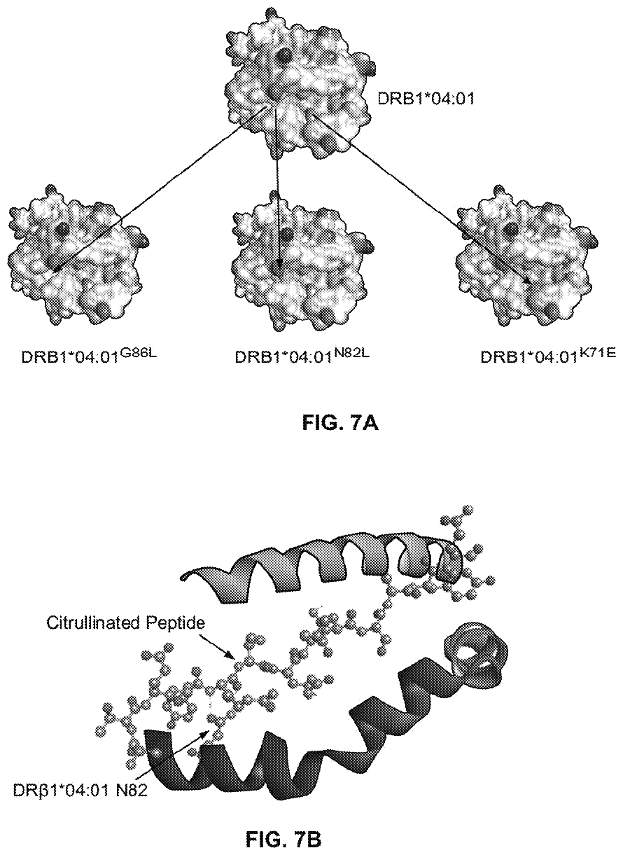

FIG. 7A presents three-dimensional protein structures of the DRB1*04:01 protein. The relative locations of the modifications G86L, N82L, and K71E are indicated. FIG. 7B presents a ribbon model of the peptide binding region of the DRB1*04:01 peptide. It shows the location of the N82 residue in relation to the binding regions of a citrullinated peptide.

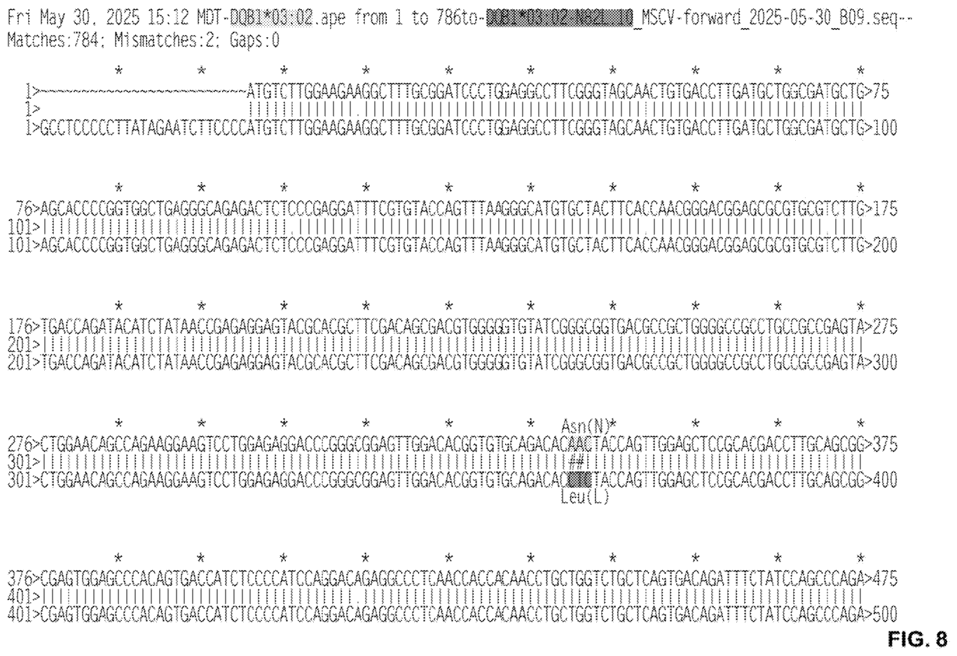

FIG. 8 presents a sequence alignment of DQB1*03:02 and DQB1*03:02^{N82L} alleles. The asparagine to leucine modification is indicated.

FIG. 9 presents the analysis of DQB1 peptide binding and T-cell activation gating strategy.

### Example 5: Editing Asparagine to Leucine at Position 82 in Type-1 Diabetes-Associated Allele DQB1*03:02 Blocks Pathogenic Peptide Binding and T-Cell Activation

The Effects of Mutating Asparagine at Position 82 for Type-1 Diabetes Associated Allele DQB1*03:02, were assessed by incubating mutant K562 cells, DQB1*03:02 and DQB1*03:02^{N82L}, with biotinylated HIP11, a hybrid insulin peptide that is presented by HLA-DQB1 and associated with the pathogenesis of Type-1 Diabetes. Unbound biotinylated peptide was washed away followed by incubation with PE-conjugated streptavidin beads. Flow cytometry was subsequently performed to determine peptide binding. As shown in FIGS. 4B and 4C, incubation of endogenous DQB1*03:02 with HIP11 shows binding to the endogenous DQB1*03:02 allele, which is ablated by the N82L edit, as evidenced by the overlapping peaks in the N82L histogram (FIG. 4B, bottom panel) compared to the distinct peaks shown for the unedited cells with the endogenous DQB1*03:02 allele (FIG. 4B, top panel). FIG. 4B presents flow cytometric histograms of HIP11 (a specific hybrid insulin peptide that is a potential autoantigen in TID) being assessed for its binding to Type-1 diabetes associated with DQ8 allele expressed in K562 cells. FIG. 4C presents a graphical representation of T-cell activation in cells expressing wild-type DQB1*03:02 or DQB1*03:02^{N82L} proteins. See also FIG. 11 which also shows that the N82L edit has limited binding of HIP11. FIG. 11 presents dot plots which show that the DQB1*03:02^{N82L} allele is expressed, and flow cytometric histograms that show that the N82L edit has limited binding of HIP11 at 100 μM in three separate cell samples.

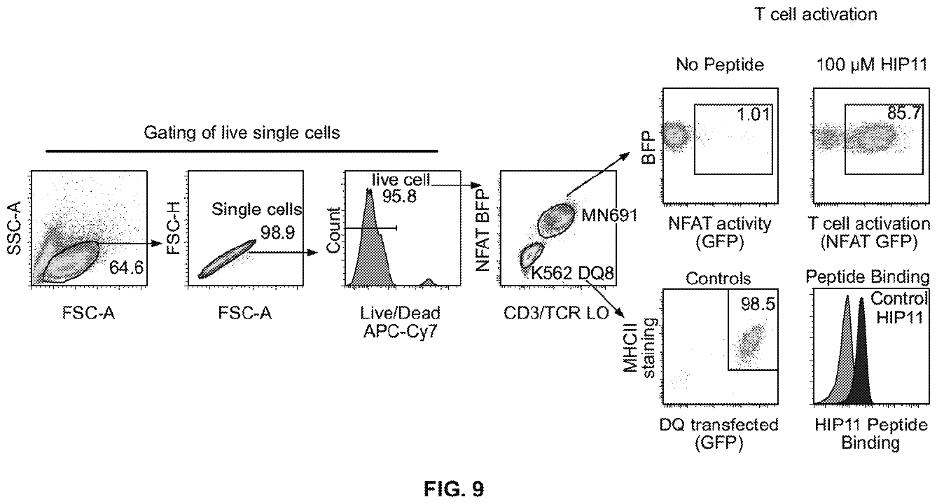

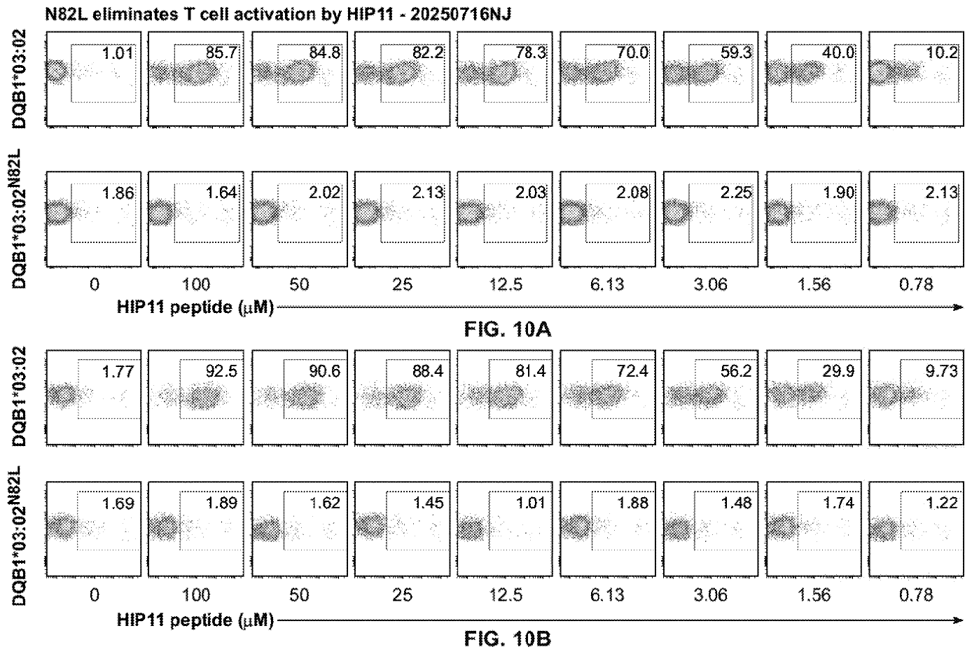

The NFAT assay adapted from Landry et al. Bio-protocol 11(02):e3883 is used to quantify T-cell activity in cells with the DQB1*03:02 allele. Flow cytometry analysis is performed to quantify T-cell activation in response to adding hybrid insulin peptide, HIP11. Analysis of DQB1 peptide binding and T-cell activation gating strategy is shown in FIG. 9. Scatterplots showing the quantified T-cell activation in cells with DQB1*03:02 and DQB1*03:02^{N82L} are shown in FIGS. 10A and 10B which show that the N82L edit eliminates T-cell activation by HIP11. FIG. 4B presents dose response curves that show T-cell activation in response to increasing HIP11 concentrations for K562 cells with the DQB1*03:02 and DQB1*03:02^{N82L} allele. The DQB1*03:02 allele shows highly active T-cell responses to HIP11 whereas there is no measurable T-cell response generated from the edited DQB1*03.02^{N82L} cells. The quantified results are summarized in Table 4 below.

TABLE 4
Peptide Binding and TCR activation for DQB1*03:02 allele N82L Edit

| | | Peptide Binding Ratio | | Peptide | TCR |
|---|---|---|---|---|---|
| Alleles | | (values over 1 are positive) | | Binding | Activation |
| and Edits | Peptide | Unedited | Edited | % Reduction | % Reduction |
| DQB1*03:02^{N82L} | HIP11 | 5 | 2 | 78% | 100% |

### Example 6: Editing Asparagine to Leucine at Position 82 in MS-Associated Allele DRB1*15:01 Blocks Pathogenic Peptide Binding

Analysis is performed to determine the association of DRB1 and DQB1 alleles in MS. 1658 MS patients are compared to 99,962 controls to compare the frequency of DRB1 and DQB1 alleles. Statistical analysis is performed by chi square test and computation of odds ratio is determined. The results shown in Table 5 below reveal that DRB*15:01 is among the most significant HLA Class II alleles associated with MS.

TABLE 5
Chi-Square Analysis on High Resolution HLA typing
reveals the most significant HLA Class II alleles
associated with Multiple Sclerosis

| | OR (95% C.I.) | p-value | Association |
|---|---|---|---|
| HLA DR alleles | | | |
| DRB1*15:01 | 1.81(1.64-2.01) | <0.0001 | Risk |
| DRB1*14:01 | 0.61(0.46-0.79) | 0.0002 | Protection |
| DRB1*07:01 | 0.80(0.71-0.90) | 0.0003 | Protection |
| HLA DQ alleles | | | |
| DQB1*06:02 | 1.72(1.55-1.91) | <0.0001 | Risk |
| DQB1*02:01 | 0.44 (0.39-0.50) | <0.0001 | Protection |
| DQB1*05:03 | 0.59(0.45-0.77) | 0.0001 | Protection |

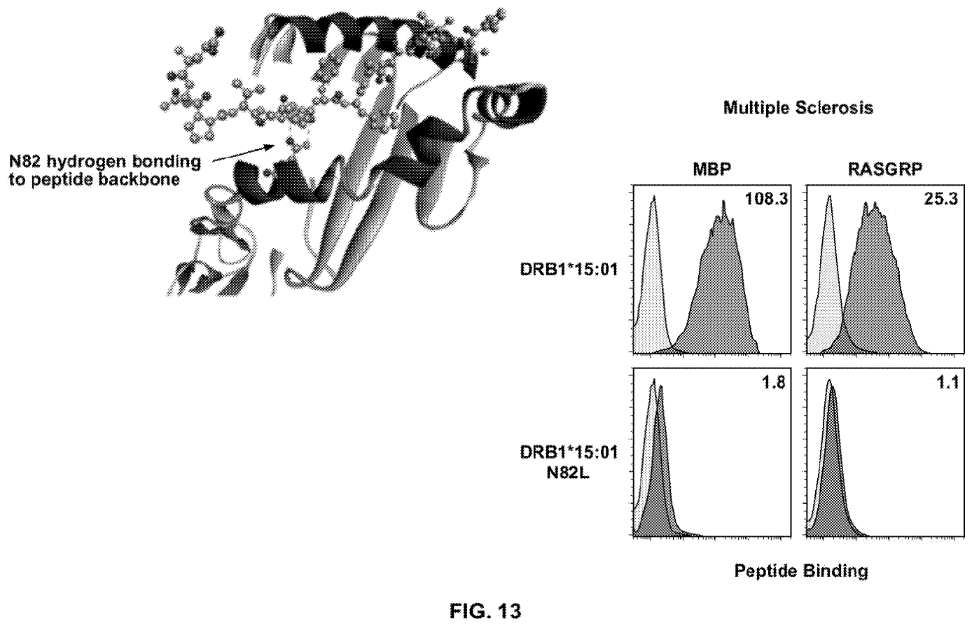

The effects of mutating asparagine at position 82 for MS-associated allele (DRB1*15.01), were assessed by incubating mutant K562 cells, DRB1*15:01 and DRB1*15:01^{N82L}, with biotinylated immunogenic proteins myelin basic protein (MBP) and RAS guanyl releasing protein 2 (RASGRP2). MBP is a key component of myelin sheath that gets degraded in MS. RASGRP2 is a target autoantigen that is expressed in the cortical grey matter and striatal neurons in the brain. Unbound peptides were washed followed by incubation with PE-conjugated streptavidin beads. Flow cytometry was subsequently performed to determine peptide binding. As shown in FIG. 13, incubation of endogenous DRB1*15:01 with either MBP or RASRGP2 shows binding to the endogenous DRB1*15:01 allele, which is ablated by the N82L edit, as evidenced by the overlapping peaks in the N82L histogram (bottom panels) compared to the distinct peaks shown for the unedited cells with the endogenous DRB1*1:.01 allele (top panels). The quantified results are summarized in Table 6.

TABLE 6
Peptide Binding and TCR activation
for DRB1*15:01 allele N82L Edit

| | | Peptide Binding Ratio | | Peptide |
|---|---|---|---|---|
| Alleles | | (values over 1 are positive) | | Binding |
| and Edits | Peptide | Unedited | Edited | % Reduction |
| DRB1*15:01 | MBP83 | 105 | 2 | 99% |
| N82L | RASGRP2 | 24 | 1 | 99% |

### Example 7: Identifying Epitopes and Antigenic Factors Involved in the Pathogenesis of Sarcoidosis

Numerous infectious and noninfectious agents have been linked to the etiology of sarcoidosis, but definitive proof that these agents are causative is lacking. For example, mycobacterial infection may be involved in the pathogenesis of sarcoidosis, and mycobacterial catalase-peroxidase (mKatG) has been identified as a candidate antigen in sarcoidosis. Drake et al. found IFN-γ-secreting cells in the blood of US sarcoidosis subjects in response to two mycobacterial proteins, mKatG and early secreted antigenic target 6 (ESAT-6). An NDPD peptide has been identified in the Applicant's laboratory as a candidate antigen and A. nidulans as a potential etiologic organism in acute sarcoidosis. Of note, these studies have demonstrated that TRAV12-1-expressing CD4⁺ T cells from the BAL of LS subjects do not respond to overlapping peptides derived from mKatG or ESAT-6. Additionally, no mycobacterial-specific CD4⁺ T cell clones have been identified from LS patients.

To identify epitopes and antigenic factors involved in the etiology of sarcoidosis, bronchoalveolar lavage fluid is collected from sarcoidosis patients and cells are sorted and selected for CD4-T cells. 10× Genomics® single cell sequencing is done to determine differentially expressed T-cell receptor (TCR) genes on single CD4 T cells. Candidate TCRs are selected based on particular characteristics that suggest they have been activated in vivo and are likely relevant to disease pathogenesis. Exemplary of hallmarks of disease relevance include preferential V gene usage, oligoclonal expansions, homologous TCRs, CDR3β and/or CDR3α, and those identified in public repositories. Next, T-cell hybridomas are generated via retroviral transduction according to standard procedures in the art. Hybridomas are TCR^{neg} and immortalized. Transfer of alpha/beta TCRs confers that TCRs specificity to the cell line. Activation by antigen is assessed in a beryllium-specificity IL-2 activation assay using various DP2-APCs. Positional scanning peptide libraries (PSLs) are used to determine stimulatory mimotopes and naturally-occurring peptides that may represent etiologic antigens in sarcoidosis. Briefly, decapeptide PSLs are used to identify mimotopes and naturally-occurring peptides from proteins for 1) a set of Bi-specific TCRs derived from the lungs of HLA-DP2-expressing chronic beryllium disease (CBD) patients and BeO-exposed HLA-DP2 transgenic mice and 2) TRAV12-1-expressing CD4⁺ T cells in the lungs of HLA-DR3-expressing LS subjects. Mimotopes are defined as peptides that differ in sequence from the naturally-occurring epitope, yet bind to the appropriate MHC molecule and are recognized by specific T cells. An advantage of PSLs for antigen discovery is that they are unbiased, consisting of mixtures that allow every possible variation of a peptide of defined length to be evaluated, thus allowing the TCR to determine the preferred amino acid at each peptide position. Identification of mimotopes using this technology resulted in the characterization of T cell epitopes for both MHCI- and MHCII-restricted T cells. Candidate TCRs derived from the BAL of DR11 sarcoidosis subjects and are expressed on hybridomas for antigen discovery studies. Table 7 lists exemplary candidate TCRs. They are distinct clonotypes but similar enough that they are likely to respond to the same antigen and are derived from multiple patients.

TABLE 7
TCRs derived from the BAL of DR11 Sarcoidosis Subjects

| DR11 | | | | | | | |
|---|---|---|---|---|---|---|---|
| Sarc | Hyb | TRAV | CDR3 Sequence | TRAJ | TRBV | CDR3 Sequence | TRBJ |
| *4879 | Clone 1 | 26-1 | CIVSFAGGTSYGKLTF | 52 | 9 | CASSLTGGYGYTF | 1-2 |
| *4879 | Clone 2 | 26-1 | CIGNAGGTSYGKLTF | 52 | 9 | CASSLSNYGYTF | 1-2 |
| 4739 | Clone 1 | 26-1 | CISQKSGGTSYGKLTF | 52 | 9 | CASSLTGGYEQYF | 2-7 |
| 4739 | Clone 2 | 26-1 | CIVSIAGGTSYGKLTF | 52 | 9 | CASSLSNYGYTF | 1-2 |
| 4661 | Clone 1 | 26-1 | CTLSLAGGTSYGKLTF | 52 | 9 | CASSVTGGYGYTF | 1-2 |

HLA-DR11/peptide tetramers are also generated to screen PBMC and BAL of HLA-DR11⁺ sarcoidosis patients to enumerate the frequency of these cells and assess how widespread the T cell specificity is among sarcoidosis patients and controls.

The above-described multistep process is an iterative process that has indicated mimotopes and naturally occurring peptides that stimulate a set of TCRs that are a focus of further downstream analysis.

A biometrical analysis is performed to identify candidate naturally occurring peptides capable of stimulating the T cell hybridomas. Scoring matrices derived from T cell hybridoma responses to peptide libraries are applied to all possible overlapping decapeptides in the SwissProt protein database to rank peptides by their predicted stimulatory potential. The database search included all species to ensure no candidates were excluded.

Greater than 150 of the top-ranked peptides were synthesized and evaluated for hybridoma stimulation. Among these, peptide BM03 produced the strongest response. This peptide is derived from non-histone chromosomal protein 6 and is an exact match expressed by numerous fungal species. Previous work investigating CD4 T cell antigen specificity in HLA-DR3-associated acute sarcoidosis (Lofgren's Syndrome) also identified a fungal peptide, from Aspergillus nidulans that stimulated pulmonary T cells from multiple patients. Together, these results suggest that fungi may contribute to the pathogenesis of diverse forms of sarcoidosis, and the HLA genes that present these antigens, i.e., HLA-DRB1*11:01 may represent amenable targets for HLA gene editing to temper T cell responses to these organisms.

### Example 8: T-Cell Hybridomas from BAL-Derived CD4 T-Cells of Sarcoidosis Subjects can Result in Significant Reduction of IL-2 Secretion in Cells with N82L Edit

A T-cell hybridoma assay is performed to measure functional response of the T-cell hybridomas. In 96-well flat-bottomed plate, the following components are combined:
5×10⁴ transfected (or transduced) T2 cells expressing wild-type or variants of DR11
2.5×10⁴ T cell hybridoma cells expressing TCRs from BAL-derived CD4 T cells of HLA-DR11⁺ sarcoidosis subjects
Naturally occurring peptides (concentrations ranging from 01.nM to 30 μM) derived from the deconvolution and biometrical analysis of decapeptide positional scanning library screens of the hybridomas and that stimulate the candidate TCRs (i.e., the TCRs believed to be disease-relevant due to their gene-segment usage, CDR3 homologies and presence in multiple DR11 sarcoidosis subjects)
Complete IMDM Medium: (10% FBS)

The samples are incubated 22-24 hours overnight and an ELISA is performed to quantify secretion of murine IL-2. The peptides to be evaluated for their activity with T2 cells expressing HLA-DR11:01 and DR11 variants are shown in Table 8.

TABLE 8

| | |
|---|---|
| | Amino Acid Sequence |
| Name Peptide | p1 p2 p3 p4 p5 p6 p7 p8 p9 p10 |

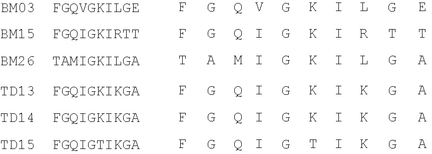

note:
all decapeptides were N-acetylated and C-amidated; differences highlighted.

T-cell hybridomas 4879-C1 and 4879-C2 express TCR-derived CD4 T cell clones isolated from the bronchoalveolar lavage of an HLA-DR11 sarcoidosis patient. Cells with the N82L edit can show significant reductions in IL-2 activity in response to BM03, BM15 and BM26 peptides. Responses to TD13, TD14, and TD15 polypeptides can also show reductions in IL-2 activity in cells with the N82L edit.

### Example 8: Effects of N82L Edit for Celiac Disease

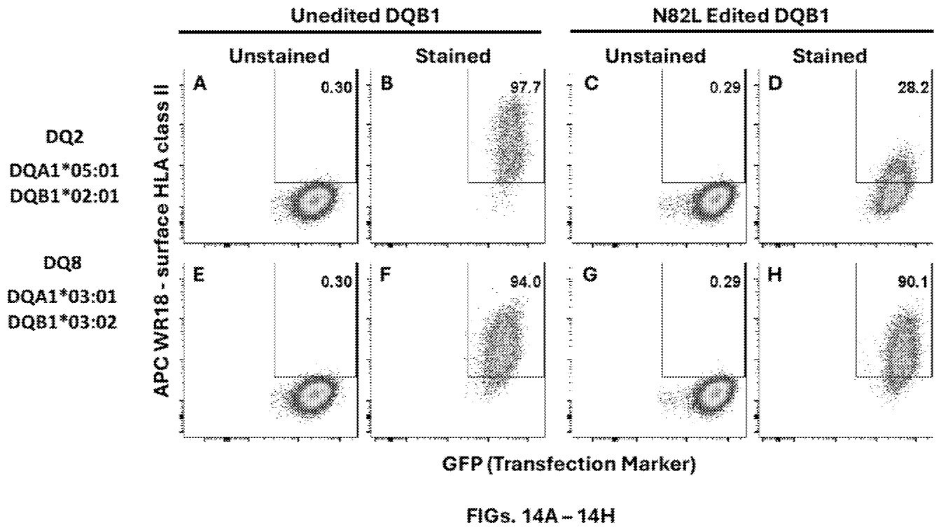

Expression of HLA class II molecules on the plasma membrane of K562 cells transfected to express the indicated DQ2 and DQ8 molecules was evaluated. FIGS. 14A-14H present representative flow cytometric plots comparing cell-surface expression of HLA-DQ molecules on the plasma membrane of K562 cells transfected to express the indicated DQ2 and DQ8 molecules. For each construct, the corresponding unstained negative controls (FIG. 14A, FIG. 14C, FIG. 14E, and FIG. 14G) are shown alongside the matched anti-DQ-stained sample (FIG. 14B, FIG. 14D, FIG. 14F, and FIG. 14H), thereby illustrating background fluorescence and the staining gate used to identify HLA-DQ-positive cells. The value shown in the upper-right corner of each plot denotes the percentage of live, transfected cells exhibiting detectable HLA-DQ surface expression. As shown, surface expression of DQ2 (FIG. 14B) is markedly diminished upon introduction of the N82L substitution (FIG. 14D). In contrast, surface expression of DQ8 (FIG. 14F) remains largely unchanged following the N82L edit (FIG. 14H), indicating an allele-dependent effect of the N82L substitution on HLA-DQ trafficking and/or stability at the cell surface.

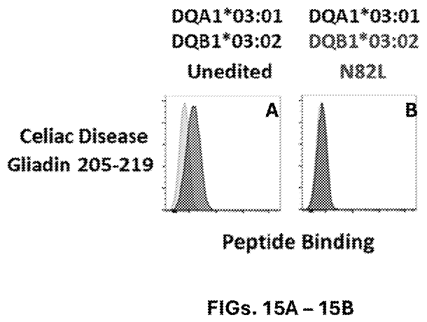

The effects of mutating asparagine at position 82 for the celiac disease-associated allele (DQB1*03:02), were assessed by incubating K562 control cells and transfected K562 cells expressing the endogenous unedited DQB1 allele or the N82L edited allele with biotinylated gliadin peptide. Gliadin is a potential autoantigen that drives celiac disease pathogenesis. Unbound peptides were washed followed by incubation with PE-conjugated streptavidin beads. Flow cytometry was subsequently performed to determine peptide binding. As shown in FIGS. 15A-15B, incubation of unedited DQ8 (DQA1*03:01/DQB1*03:02) shows a shift in peptide binding (dark grey histograms in FIG. 15A) compared to the negative controls (light grey histograms in FIG. 15A). This effect is ablated by the N82L edit, as evidenced by the overlapping peaks in the N82L histogram (FIG. 15B). The quantified results are summarized in Table 9.

TABLE 9
Gliadin peptide binding for the DQ8 (DQA1*03:01/DQB1*03:02)
unedited and DQB1*03:02 N82L edited alleles

| Table 9 | | | | Peptide |
|---|---|---|---|---|
| Alleles | | Peptide Binding Ratio | | Binding |
| and Edits | Peptide | Unedited | Edited | % Reduction |
| DQA1*03:01 | Native | 2 | 1 | 89% |
| DQB1*03:02 | Gliadin | | | |
| N82L (DQ8) | | | | |

The data used to calculate the peptide binding ratios were generated from raw MFI values shown in Table 10. The mean fluorescence intensity (MFI) in the PE channel detected by flow cytometry is shown for the untransfected control cells (Controls) and transfected cells (Unedited or Edited). Peptide binding ratios were calculated by dividing the Raw MFI for HLA expressing cells by the MFI detected from Controls. To estimate the % reduction in peptide binding, the Control MFIs were subtracted from Unedited and Edited MFIs and then the percent change was calculated.

TABLE 10
Raw mean fluorescence intensity (MFI) peptide binding data

| Indication, Alleles | | | | |
|---|---|---|---|---|
| and Edits | Peptides | Controls | Unedited | Edited |
| Rheumatoid | Collagen | 113 | 5183 | 91 |
| Arthritis | Native alpha-Enolase | 197 | 454 | 199 |
| DRA1*01:01 | Citrullinated alpha-Enolase | 146 | 4793 | 122 |
| DRB1*04:01 | Native Vimentin | 116 | 160 | 89 |
| N82L | Citrullinated Vimentin | 403 | 10181 | 1082 |
| | HA | 324 | 4036 | 1740 |
| Rheumatoid | Collagen | 113 | 5183 | 123 |
| Arthritis | Native alpha-Enolase | 197 | 454 | 206 |
| DRA1*01:01 | Citrullinated alpha-Enolase | 146 | 4793 | 167 |
| DRB1*04:01 | Native Vimentin | 116 | 160 | 105 |
| N82M) | Citrullinated Vimentin | 403 | 10181 | 303 |
| | HA | 324 | 4036 | 339 |
| Multiple Sclerosis | MBP83 | 407 | 42517 | 322 |
| DRA1*01:01 | RASGRP2 | 629 | 14367 | 123 |
| DRB1*15:01 | | | | |
| N82L | | | | |
| Type 1 Diabetes | Hybrid Insulin Peptide 11 | 103 | 485 | 175 |
| (DQ8) | | | | |
| DQA1*03:01 | | | | |
| DQB1*03:02 | | | | |
| N82L | | | | |
| Celiac (DQ8) | Native Gliadin | 82 | 174 | 92 |
| DQA1*03:01 | | | | |
| DQB1*03:02 | | | | |
| N82L | | | | |

### Example 9: Effects of N82L Edit for Neuromyelitis Optica

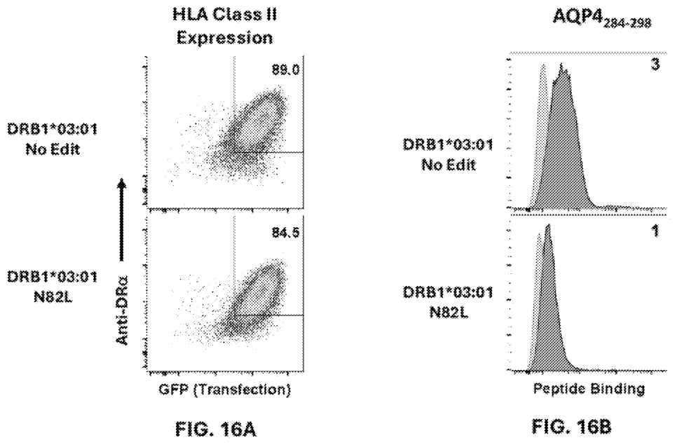

The effects of mutating asparagine at position 82 for the NMO allele (DRB1*03:01), were assessed by incubating control cells and cells expressing the endogenous unedited DRB1*03:01 allele or the N82L edited allele with biotinylated aquaporin 4 peptide. This peptide is a known autoantigen that drives nerve destruction in neuromyelitis optica. Unbound peptides were washed followed by incubation with PE-conjugated streptavidin beads. Flow cytometry was subsequently performed to determine if the N82L edit impacted surface expression of a functional class II molecule or peptide binding. FIGS. 16A and 16B present flow cytometric analysis of surface MHC expression in T2 lines expressing the alleles shown and peptide binding, respectively. FIG. 16B compares binding of an aquaporin 4 peptide by T2 cells expressing no HLA class II on the surface (light histogram in FIGS. 16B, top and bottom panels), DRB1*03:01 transfected cells with no edits (dark histograms in FIG. 16B, top panel), and DRB1*03:01 transfected cells with N82L edits (dark histograms in FIG. 16B, bottom panel).

As shown in FIGS. 16A and 16B, incubation of peptide in cells expressing the unedited molecule shows a shift in peptide binding (dark grey histogram in FIG. 16B, top panel) compared to the negative controls (light grey histogram in FIG. 16B, top panel). This effect is ablated by the N82L edit, as evidenced by the overlapping peaks in the N82L histogram (FIG. 16B, bottom panel). Numbers in the top right corner of each panel indicate the fold increase in peptide binding vs controls for each allele. The raw data and quantified results are summarized in Table 11. This is pilot data and experiments are currently being repeated.

TABLE 11
Raw AQP4284-298 peptide binding signal (geometric mean fluorescence intensity), peptide
binding ratios and % reduction in normalized GMFI by T2 cell lines expressing no DRB1
(CTL) or transfected to express the unedited and N82L edited DRB1*03:01 allele.

| | | | | | Peptide Binding Ratio | | |
|---|---|---|---|---|---|---|---|
| NMO Peptides (Pilot Data) | | | | | (values over 1 are | | Peptide |
| Alleles and | | Peptide Binding GMFI | | | positive) | | Binding GMFI |
| Edits | Peptide | CTL | Unedited | Edited | Unedited | Edited | % Reduced |
| DRB1*03:01 | AQP4₂₈₄₋₂₉₈ | 139 | 394 | 186 | 3 | 1 | 82% |
| N82L | | | | | | | |

Calculations of peptide binding ratios were identical to those described previously except that geometric mean more accurately reflected the histogram data.

While multiple embodiments are disclosed, still other embodiments of the presently disclosed concepts, compounds, compositions, methods, processes, systems, and therapies will become apparent to those skilled in the art from the following detailed description. As will be apparent, the present disclosure is capable of modifications in various obvious aspects, all without departing from the spirit and scope of the present disclosure. Accordingly, the detailed description is to be regarded as illustrative in nature and not restrictive.

Although the present disclosure has been described with a certain degree of particularity, it is understood the disclosure has been made by way of example, and changes in detail or structure may be made without departing from the spirit of the disclosure as defined in the appended claims.

1. A recombinant HLA protein comprising:
an amino acid sequence having at least 95% sequence identity to a wild-type HLA protein; and
an amino acid substitution at position 82 from asparagine to a non-polar amino acid selected from alanine, valine, leucine, proline, methionine, tryptophan, glycine, isoleucine, or phenylalanine, wherein the recombinant HLA protein has an antigen-binding affinity different from that of the wild-type HLA protein.

2. The recombinant HLA protein of claim 1, wherein the non-polar amino acid is leucine.

3. The recombinant HLA protein of claim 1, wherein the non-polar amino acid is methionine.

4. A method of preparing edited hematopoietic stem and progenitor cells (HSPCs) from a subject, the method comprising:
isolating hematopoietic stem and progenitor cells (HSPCs) from the subject;
editing an HLA allele in genomic DNA of the HSPCs by replacing nucleotides encoding asparagine at amino acid residue 82 with nucleotides encoding a non-polar amino acid; and
selecting, isolating, and optionally expanding the edited HSPCs.

5. An ex vivo method of treating or preventing an HLA class II-associated autoimmune disease in a subject, the method comprising:
administering to the subject the edited HSPCs of claim 4, thereby treating or preventing the autoimmune disease in the subject.

6. The ex vivo method of claim 5, wherein the non-polar amino acid is selected from alanine, valine, leucine, proline, methionine, tryptophan, glycine, isoleucine, or phenylalanine.

7. An in vivo method of treating or preventing an HLA class II-associated autoimmune disease in a subject, the method comprising:
administering to a subject a delivery vehicle comprising one or more of a nucleic acid, a protein, and a cofactor to effect editing of the HLA allele;
editing the HLA allele in a genomic DNA at position 82 by replacing nucleotides encoding asparagine with nucleotides that encode a non-polar amino acid; and
thereby treating or preventing the autoimmune disease in the subject.

8. The in vivo method of claim 7, wherein the non-polar amino acid is one of alanine, valine, leucine, proline, methionine, tryptophan, glycine, isoleucine, or phenylalanine.

9. The method of claim 5, wherein the disease is rheumatoid arthritis, diabetes mellitus type 1, multiple sclerosis, or celiac disease.

10. The method of claim 5, wherein the disease is rheumatoid arthritis.

11. The method of claim 5, wherein the disease is diabetes mellitus type 1.

12. The method of claim 5, wherein the disease is multiple sclerosis.

13. The method of claim 5, wherein the disease is neuromyelitis optica.

14. The method of claim 7, wherein the delivery vehicle comprises one or more of a viral vector, a lipid nanoparticle, or a virus-like particle.

15. The method of claim 7, wherein the delivery vehicle comprises a BactPac™ delivery vector.

16. A gene-editing delivery vehicle for use in treating autoimmune diseases or conditions comprising:
a guide RNA sequence complementary to a target nucleic acid sequence within an HLA allele;
a Cas9 protein or a similar endonuclease; and
a template nucleic acid comprising a portion of the HLA allele with the nucleotides encoding asparagine at position 82 replaced with nucleotides encoding a non-polar amino acid.

17. The gene-editing delivery vehicle of claim 16, wherein the delivery vehicle comprises one or more of a viral vector, a lipid nanoparticle, or a virus-like particle.

18. The gene-editing delivery vehicle of claim 16, wherein the delivery vehicle comprises a BactPac™ delivery vector.

19. The gene-editing delivery vehicle of claim 16, wherein the HLA allele is an HLA class II allele associated with an autoimmune disease.

20. The gene-editing delivery vehicle of claim 16, wherein the non-polar amino acid is leucine or methionine.
